(19) United States
(12) Patent Application Publication
(10) Pub. No.: US 2019/0226004 A1
(43) Pub. Date: Jul. 25, 2019

(54) MULTI-LEVEL, LABORATORY-BASED SURVEILLANCE SYSTEM FOR DETECTION OF INTRAOPERATIVE "ESKAPE" BACTERIAL PATHOGENS FOR HCAI PREVENTION

(71) Applicant: RDB BIOINFORMATICS, LLC, Omaha, NE (US)

(72) Inventor: Randy W. Loftus, Iowa City, IA (US)

(21) Appl. No.: 16/258,319

(22) Filed: Jan. 25, 2019

Related U.S. Application Data
(63) Continuation of application No. PCT/US17/26557, filed on Apr. 7, 2017.
(60) Provisional application No. 62/320,192, filed on Apr. 8, 2016.

(51) Int. Cl. C12Q 1/04; G16H 50/70; C12Q 1/02; G16B 99/00; G16B 10/00; G16B 50/00; G16B 40/00

(52) U.S. Cl. CPC C12Q 1/04; G16H 50/70; C12Q 1/025; G16B 40/00; G16B 10/00; G16B 50/00; G16B 99/00

(57) ABSTRACT

The present invention provides systems and methods for surveillance, diagnosis, and evaluation of high risk bacterial transmission events. The systems and methods utilize software and computational systems that automate identification, surveillance, and communication. The invention further includes archival systems for use in the systems and methods that compile bacterial isolates linked to information about patients, pre-operative, intra-operative, or post-operative arenas, healthcare providers, and the like.

### CROSS REFERENCE TO RELATED APPLICATION

This application is a by-pass Continuation Application of PCT/US2017/026557, filed Apr. 7, 2017, which claims priority under 35 U.S.C. § 119 to provisional application Ser. No. 62/320,192, filed Apr. 8, 2016, all of which are herein incorporated by reference in their entirety.

### FIELD OF THE INVENTION

The present invention is directed to surveillance systems and methods for the diagnosis and evaluation of high risk bacterial transmission events.

### BACKGROUND OF THE INVENTION

Healthcare-associated infections (HCAIs) are a devastating and persistent problem, affecting one in every twenty-five patients admitted to hospitals today. Bacterial pathogens have evolved to acquire a multitude of genetic traits that favor bacterial infection, including increased transmissibility, increased virulence, and increased antibiotic resistance. As a result of this evolutionary triad, medicine has entered the “post antibiotic era” where antibiotics are no longer as effective in treating infections when they develop. As such, prevention of bacterial transfer, a root cause of infection development, is of paramount importance, and it must be addressed quickly. This is especially true for bacterial pathogens such as S. aureus, Enterococcus faecium, Klebsiella pneumoniae, Pseudomonas aeruginosa, Acinetobacter baumanni, and Enterobacter spp. organisms that are particularly successful in causing patient harm. These ESKAPE pathogens are the primary target of the documented claims, as without focused efforts to target these pathogens, great patient harm will continue.

The current art is unsuccessful at controlling these pathogens. In 2011, K. pneumoniae, now known as a “super bug,” killed several patients at a leading National Institutes of Health (NIH) research hospital despite aggressive attempts to prevent its spread. The organism was transferred from an infected patient to 18 additional patients, killing 6 (33%) of those affected by the bacterial transfer. The same organism affected another patient at the same hospital a year later. This outbreak clearly conveyed that even the most advanced hospitals today are not equipped to understand, and therefore to intervene in, the deadly process of bacterial transfer.

This is a significant healthcare issue that has captured national attention, culminating in an executive order put forth by the White House in September of 2014. In this executive order, President Obama demands that the NIH work with investigators to bring advanced technologies, such as improved surveillance, next generation sequencing, and rapid, point-of-care diagnostics to the patient bedside to improve basic preventive measures such as hand hygiene, patient skin decolonization, and environmental cleaning in order to prevent bacterial spread. This goal, to prevent infections before they develop in order that at least some antibiotics remain effective for the future, continues to be a major focus of the White House.

Patient colonization with deadly pathogens is a basic measure in need of improvement. Patients frequently arrive to the intraoperative environment with skin surfaces colonized with major bacterial pathogens, and less than twenty percent of patients are effectively decolonized preoperatively. Colonized patient skin surfaces serve as a major bacterial reservoir in the operative environment, one that often participates in vertical bacterial transmission leading to infection in the patient. Surprisingly, patient-derived strains are transmitted to subsequent patients undergoing procedures on the same day in the same operating room, again leading to HCAI development. Thus, patient skin colonization is a major factor impacting other patients undergoing care in the same arena (i.e., between operative cases), is the main source of Staphylococcus aureus origin and transmission, and is the main source of 30-day postoperative infections (both between and within operative cases) from S. aureus. While patient colonization contributed partially to bacterial transmission within the environment, it also significantly contributed to endogenous infection (in 83% of cases). Improvement in patient decolonization is a critical need.

The operating room environment (ORE) includes health care tools and surfaces used within the anesthesia work environments (AWE), air, and even anesthesia health care providers themselves. It has long been known that syringes and intravascular catheters can become contaminated directly via bacterial contamination of the provider's hands or indirectly during connection to patient IV tubing. In 1974, Blogg et al. reported that syringes can become contaminated with bacterial pathogens after a single use, thereby providing a plausible mechanism for the bacterial contamination of propofol vials later linked to cases of severe sepsis and a series of Staphylococcus aureus bloodstream infections occurring in patients undergoing electroconvulsive therapy. Laryngoscope blades and handles are contaminated with blood and mucus after use and standard disinfection procedures. Residual contamination of these airway devices associated with suboptimal disinfection practices has been linked to infectious outbreaks. Additional work has con-firmed the need for better disinfection of laryngoscope handles in today's ORE and AWE. Contamination of anesthesia machine surfaces with blood, mucus, and bacterial organisms after standard cleaning processes was first characterized in the 1960s and subsequently confirmed. Many of these disinfection practices are still used today. Numerous early studies reported the ability of recovered bacterial pathogens to survive on anesthesia equipment for several days and to serve as potential sources of infection. In addition, residual surface contamination was identified as a possible link to a cluster of follicular tonsillitis infections, all occurring in the same postoperative week. Other reports have documented an association of residual contamination of the anesthesia machine circuit and Ambu-bag with outbreaks of Pseudomonas aeruginosa respiratory infections. Bacterial contamination of the anesthesia machine circuit is also important to consider as a risk factor for bacterial transmission in the AWE. Early work identified an association between combined preoperative decontamination of the external surface of anesthesia machine circuits and use of new absorbers with a reduction in postoperative pneumonia, and concluded that contaminated anesthesia machines can indeed transmit bacteria to patients. Thus, improvement in intraoperative cleaning is also a critical need to prevent the intraoperative spread of dangerous bacteria.

Additional work has examined bacterial contamination on the hands of anesthesiologists during general anesthesia, and found that they were heavily contaminated with bacterial pathogens throughout all phases of anesthesia care. These findings are concerning given that anesthesia providers have been shown to be particularly noncompliant with hand hygiene. Ninety-five percent of anesthesiologists surveyed in one study reported washing their hands after caring for “high-risk” patients, but only 58% washed their hands in “low-risk” situations. More recent observational work has shown that lapses in hand hygiene compliance occur frequently in today's OR. Furthermore, these lapses often involve failure to wash hands before and/or after aseptic tasks involving line insertions, bronchoscopy, or even after blood exposures. Thus, when taken together, these data support the links between anesthesia providers and postoperative infectious outbreaks reported as early as the 1960s. Thus, improvement in intraoperative hand hygiene is also a critical need to prevent the spread of dangerous bacteria.

Specific strains and/or strain characteristics of pathogenic organisms make them more likely to resist decontamination procedures or eradication by antibiotics administered during the perioperative period, and thus are more likely to be transmitted to other patients or to the patient's surrounding environment (“patient nest”) during the process of patient care, are more likely to lead to HCAI development and/or mortality, and are more likely to lead to hospital readmission and associated increases in the cost of patient care. This is in part due to the ability of these organisms to form institutional reservoirs that if left undetected, continually affect patients over time with repeated exposure. The ability to detect and specifically prevent, target, the spread of these “superbugs” is a critical need.

Unfortunately, while there are several critical needs, current technology is insufficient to address these issues. This is because in a continually changing clinical environment that is repeatedly contaminated with evolving bacterial pathogens that are themselves changing spontaneously or in response to preventive measures, a system must continually characterize the epidemiology of bacterial transmission in order to be effective. Key reservoirs of origin, modes of transmission, portals of entry and exit, transmission locations, and strain characteristics driving the success of the causative organism of infections must be understood, updated frequently to keep pace with clinical and bacterial evolution, and proactively targeted to prevent institutional reservoir development, ongoing bacterial spread, and patient infection and death.

As stated above, these goals can only be achieved with dynamic surveillance. Existing surveillance of infection is retrospective and static, representing a cross section in time, focusing on mining of existing hospital data. There are no existing systems that continually monitor bacterial transmission events in any given hospital setting, and specifically, there are no systems that are designed to proactively and dynamically track the spread of the most dangerous bacteria affecting patients undergoing surgery today, ESKAPE bacteria. Without such technology, it is very difficult to understand the cause of postoperative HCAIs, the spread of bacterial virulence factors leading to infectious outbreaks, or to keep pace with bacterial response(s) to preventive measures implemented in response to such issues. For example, interventions can quickly select for bacterial strains that have already developed the capacity to circumvent the prescribed hospital defense, fueling the very problem the intervention was intended to address. As such, the status quo of infection surveillance results in delayed, and often single interventions that are prone to failure, unable to generate sustained effects, and potentially fueling the problem.

The system can provide real-time, continual surveillance of actual ESKAPE bacterial transmission events, using a unique, multilevel, systematic-phenotypic-genotypic surveillance system tied to a novel software platform that brings key information pertaining to ESKAPE reservoirs of origin, portals of entry/exit, modes of transmission, and bacterial strain characteristics to the end user via meaningful reports that drive continual, proactive, evidence-based improvements infection control measures. System software implementation impacts patients in the preoperative, intraoperative, and postoperative period, integrating high-risk patient identification modeling with preoperative ESKAPE transmission, perioperative ESKAPE tracking, and postoperative infection tracking in order that an infection control perioperative team can implement rapid, plan-do-study-act cycles involving multimodal improvement strategies. The real-time, proactive, continual nature of the system allows the end user to keep pace with the evolution of bacterial pathogens, and ultimately, to generate sustained reductions in HCAIs.

It is therefore an objective of the present invention to provide surveillance technology specifically designed to detect the most dangerous bacteria in today's operating room environments (ESKAPE), to characterize the epidemiology of ESKAPE transfer events, to generate meaningful reports via use of innovative software, and to implement the system components in an integrated platform in order to generate real-time, proactive improvements in basic preventive measures to maximally attenuate perioperative ESKAPE transmission and subsequent infection development. This technology leverages next generation sequencing and the development of rapid, point-of-care diagnostics, and in parallel, it identifies molecules (biomarkers) that explain hyper transmissible, hyper virulent, and hyper resistant bacterial characteristics. As such, it provides the platform for the development of novel diagnostics that can be matched with novel disinfection and therapeutic agents in order to target the most deadly bacteria.

It is a further objective of the present invention to extend the intervention from the operating room to the hospital floors and to the intensive care unit environment to reduce bacterial transmission to provide systems and methods that produce hospital-wide reductions in bacterial transmission.

It is a further objective of the present invention to improve patient decolonization in preoperative arenas and to identify bacterial strains that are more likely to be transmitted and/or to cause infection by identifying patient carriers in preoperative arenas, identifying environmental components that lead to transmission events during patient care, and to identify factors that lead to transformation of less virulent to more virulent microbes.

It is a further objective of the present invention to provide systems and methods that produce hospital-wide reductions in bacterial transmission.

It is a further objective of the present invention to generate a large bacterial archive of clinically relevant pathogens (hyper transmissible, resistant and virulent) that can be used to identify new genetic traits with functional consequences that can serve for development of novel diagnostics matched with novel therapeutics.

It is a further objective of the present invention to utilize computational systems to match high risk patients with low risk environments in operating rooms and other hospital settings.

It is a further objective of the present invention to create a genetic fingerprint for hospitals so that we can more accurately determine where infections are coming from, handle those infections, and appropriately rank hospitals in terms of their ability to prevent infections.

It is a further objective of the present invention to provide systems and methods for long term care facilities, nursing homes, and the food industry to track and prevent food borne illnesses.

It is a further objective of the present invention to provide systems and methods for the military to prevent spread of disease in close quarters.

It is a further objective of the present invention to provide systems and methods for the military to use the bacterial archive to identify and to understand bacterial pathogens that are more likely to survive hostile threats such as temperature, acidity, etc.

### BRIEF SUMMARY OF THE INVENTION

The present invention provides benefits over existing systems for and methods of monitoring and surveillance of bacterial transfer because it is the only validated system for the operating room environment, it specifically targets ESKAPE pathogens (Enterococcus faecium, S. aureus, Klebsiella pneumoniae, Acinetobacter baumanii, Pseudomonas aeruginosa, and Enterobacter sp.), it is proactive and dynamic as opposed to retrospective, it leverages the platform of temporal association, it continually catalogs bacterial pathogens identifying biomarkers for hyper transmissible, resistant, and virulent pathogens, providing the substrate for ongoing development of diagnostics that can be matched to therapeutic measures, it includes a systematic perioperative bacterial reservoir collection system linked to a laboratory software program that automates the identification of ESKAPE transmission events, it uses preoperative identification of patients at increased risk of HCAI development to fuel targeted screening for ESKAPE pathogens that subsequently guides perioperative ESKAPE surveillance and tracking, and it utilizes innovative surveillance software and comprehensive implementation plans to bring next generation sequencing to the patient bedside in a cost-effective manner to improve basic preventive measures.

In an embodiment, the invention is directed to a method of preventing bacteria transmission such that infection is reduced. The method preferably comprises identifying patients at risk of developing postoperative infections, providing one or more patients identified as being at risk for a particular post-operative infection with one or more treatments capable of treating or preventing said infection; obtaining physical samples from environment, patient, hand and other samples from the identified high risk patient, operating room, and hospital ward. Preferably, the identify step in the method comprises obtaining infection risk information from one or more pre-operative, intra-operative, or post-operative arenas; screening patients for development of or as having a high risk for development of infection, wherein said screening comprises processing the infection risk information; and identifying one or more patients as being at risk for a particular post-operative infection based on said screening.

A preferred embodiment of the invention is also found in systems for screening patients for development or having a high risk of development of infection. Preferably the system comprises an information system in one or more of a pre-operative arena, an intra-operative arena, and/or a post-operative arena, wherein the information system automatically processes patient demographic information; a laboratory-based surveillance system; a computational system for identifying bacterial transmission events, the epidemiology of bacterial transmission events, patients that become infected, whether bacterial transmission events are linked to infection development, and bacterial isolates that are hyper transmissible, hyper virulent, and/or hyper resistant to antibiotic therapy (clinically relevant bacterial pathogens); and a database comprising bacterial isolate identities and bacterial isolate traits, wherein database is linked by an interface for communicating with a computing device to provide information relating to one or more of the other systems.

While multiple embodiments are disclosed, still other embodiments of the present invention will become apparent to those skilled in the art from the following detailed description, which shows and describes illustrative embodiments of the invention. Accordingly, the figures and detailed description are to be regarded as illustrative in nature and not restrictive. Reference to various embodiments does not limit the scope of the invention.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1 shows a strategic process diagram of targeted measures according to an exemplary embodiment of the invention. The process targets three different potential reservoirs of pathogens (patients, provider hands, and the surrounding environment) and downstream catheter care to attenuate bacterial contamination of high-risk intravascular devices.

FIG. 2 shows time to positivity for 6736053 vs. 6736153 MRSA strains according to an exemplary embodiment of the invention.

FIGS. 3A-3D show a computational predictive modeling system to identify patients at risk of developing postoperative infections according to an exemplary embodiment of the present invention.

FIG. 4 shows a computational process of detecting patients who develop infections and/or require the acquisition of patient cultures for workup of infection according to an exemplary embodiment of the invention.

FIGS. 5A-5B show an overall data flow, input, and output according to an exemplary embodiment of the invention. The overall data flow shown is complete according to the present invention, from the hospital EMR application to the internal database, including collection of patient data, bacterial reservoir collection, data processing, and reporting. Discrete elements of the overall process is depicted in FIGS. 6 through 13.

FIG. 6 shows EMR collection data flow according to an exemplary embodiment of the invention. Information is obtained from the Hospital EMR system through collecting procedure schedules, calculating patient risk (defined by risk of healthcare-associated infection development) and assigning a risk score, creating a subset of high risk patient population defined by a risk-score threshold, and saving this information to the internal derived database.

FIG. 7A shows reservoir collection data flow according to an exemplary embodiment of the invention. The data flow includes the systematic reservoir collection process that shows the previously validated collection process defined for the operating room, a model that involves previously defined sampling sites that are now proven representatives of patient, environment (including air/equipment), and provider reservoirs, a previously tested process for sampling of those defined sites that generates the highest possible bacterial yield, and an ordered sample collection process that generates a powerful platform of temporal association that aides later algorithms for processing of reservoir isolates collected.

FIG. 7B is a greyscale image of an exemplary red arrow diagram provided by the system depicting time sequence from case 1 to case 2 in the same operating room, filtered by Biofilm top 25%, showing patient source, leading to attending hand and on to patient 2, through attending hands and on to the dial.

FIG. 7C is a greyscale image of an exemplary bar chart provided by the system showing pathogen events over time grouped by class of ESKAPE pathogen, and a red arrow diagram depicting time sequence from case 1 to case 2 in the same operating room, filtered by top 5 transmissible pathogens. Transmissions most commonly occur from attending hand, to the patient, through to other hands (not on list) onto attending hands in case 2 and eventually to the valve.

FIG. 7D is a greyscale image of an exemplary red arrow diagram provided by the system depicting time sequence from case 1 to case 2 in the same operating room, filtered by Pseudomonas. Transmissions most commonly occur from Attending hand through the patient, to other hands (not on list) and then to attending hands, and on to resident hands and to the patient in case 2.

FIG. 7E is a greyscale image of an exemplary red arrow diagram provided by the system depicting time sequence from case 1 to case 2 in the same operating room, filtered by Chlorhexidine resistance top 25%. Transmissions most commonly occur starting with CRNA hands to the patient, and on to the dial, through the patient in case 2.

FIG. 7F is a greyscale image of an exemplary red arrow diagram provided by the system depicting time sequence from case 1 to case 2 in the same operating room, filtered by all pathogens involving stopcocks (LE).

FIG. 8 shows post-procedure patient data flow. This overall process uses a predefined set of criteria (FIG. 4) to automate an otherwise extremely laborious process of identifying patients that undergo care and ultimately develop infection. This process is linked to the patient encounter, case-log-identification number, which is linked to all the data from processes 1 and 2. It is repeated and reported every 5 days following surgery for a given patient encounter in order that in the case of 1 or more positive criteria [(fever (yes/no), patient culture (yes/no), anti-infective order (yes/no), and/or patient culture (yes/no)], a full chart review is conducted by the infection control team to determine if the patient suffered from one or more HCAIs according to National Healthcare Safety Network (NHSN) definitions. The determinations are fed back into the system, linked by case-log identification number.

FIG. 9 shows the data flow for the bacteria success reporting. This process identifies epidemiologically-related bacterial transmission events involving all intraoperative bacterial pathogens (true and potential) with a primary focus on ESKAPE pathogens (Enterococcus faecalis, S. aureus, Klebsiella pneumoniae, Acinetobacter baumannii, Pseudomonas aeruginosa, and S. aureus phenotypes P and H, Enterococcus phenotypes E5 and E7, the top 5 transmitted gram negative genera in today's operating room environments, and any additional pathogens that are identified by the system as circumventing current preventive measures). Bacterial pathogens involved in epidemiologically-related transmission events are then analyzed to determine if they are linked to or associate with bacterial cultures and/or NHSN-defined HCAIs defined in process 3.

FIG. 10 shows the data flow from the internal private invented database, as well as the internal lab processing.

FIG. 11 shows process 5.2, the internal lab process to analyze DNA, and return the results to the private internal invented database. The goal of process 5.2 is to process bacteria DNA, submit it to the internal private invented database, and to build custom, rapid, point-of-care diagnostics for structural variants and those structural variants determined to be the most clinically relevant (refined).

FIG. 12 shows further analysis of the bacteria DNA to discover more precise measurements of unique bacteria, and further narrowing the scope of possible transmission events. This process analyzes the impact of structural variants on the process of bacterial transmission. In addition to exploring new and better therapies to treat an infection, this process offers the opportunity to inhibit bacterial virulence and transmissibility as well.

FIG. 13 shows next generation sequencing and whole genome analysis of clinically relevant pathogens. Sequences generated by this process are first used to compare epidemiologically-related transmission events at the nucleotide level in order to identify clonal events defined when 2 or more pathogens that are epidemiologically-related also have >95% whole genome similarity and the same multi-loci sequence testing results. Clonal transmission events are then used to map institutional reservoirs defined by the presence of the same organism for greater than 7 days in a given environment

FIG. 14 Executive Summary. Shows overall workflow from the institution perspective including teams involved from Pre-surgery, pen-operative, and post-surgery monitoring. The institution uses the RDB private database to guide the process detailed in diagrams.

FIGS. 15A-C shows a High-Risk operating room Team Surveillance Workflow with the operating room process in detail including reservoir collection for case 1 (high-risk) and case 2 (following procedure).

FIGS. 16A-D show an overall workflow including the institution processes from pre-anesthesia/pre-surgery, through the operating room and includes reporting for patient and infection monitoring.

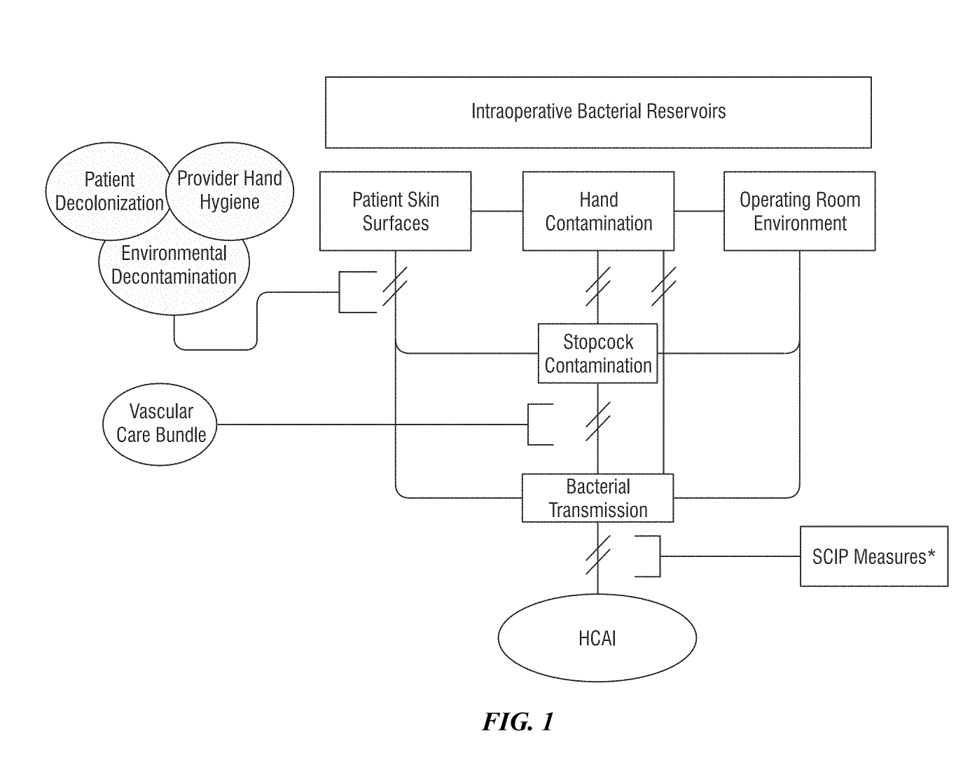

The figures represented herein are not limitations to the various embodiments according to the invention and are presented for exemplary illustration of some embodiments of the invention.

### DETAILED DESCRIPTION OF THE INVENTION

The following definitions and introductory matters are provided to facilitate an understanding of the present invention.

Numeric ranges recited within the specification, including ranges of “greater than,” “at least,” or “less than” a numeric value, are inclusive of the numbers defining the range and include each integer within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range.

The singular terms “a”, “an”, and “the” include plural referents unless context clearly indicates otherwise. Similarly, the word “or” is intended to include “and” unless the context clearly indicate otherwise. The word “or” means any one member of a particular list and also includes any combination of members of that list.

The term “Bug ID” refers to the group of codes assigned to a bacteria strain with unique phenotypic properties. The unique combination is linked to identical results throughout the system.

### Predictive Modeling to Identify Patients at Risk of Developing Postoperative Infections

The present invention includes methods for preventing post-operative infections. In one aspect, the methods involve identifying patients at risk of developing postoperative infections. In a further aspect, the identification of such patients comprises obtaining infection risk information from one or more pre-operative, intra-operative, or post-operative arenas. Pre-operative arenas include, but are not limited to, one or more of primary care offices, such as surgical, preoperative screening, pre-anesthesia testing, and/or quick care outpatient units, emergency departments, intensive care units, and/or hospital wards. Intra-operative arenas include, but are not limited to, surgical suites, operating rooms, and anesthesia work environments. Post-operative arenas include the hospital ward, post anesthesia care unit environment, same day anesthesia, and/or the intensive care unit. Infection risk information also includes information obtained from a patient and/or healthcare providers in any of the pre-, intra-, or post-operative arenas, as well as any instrumentation, tools, or equipment, including, for example, health care tools, scalpels, saws, forceps, clamps, surfaces, tubing, syringes, vials, syringe connection ports, catheters, and the like.

In preferred embodiments, the present invention is directed towards several components including; 1) a system for perioperative bacterial reservoir collection, 2) linkage of reservoir collection to a laboratory guidance system for automated detection of epidemiologically-related and clonal bacterial transmission events, 3) methodology for characterizing and reporting the epidemiology of ESKAPE bacterial transmission in order to bring whole genome analysis to the patient bedside to improve patient care via meaningful reports, 4) methodology for continually identifying and cataloging biomarkers for ESKAPE bacterial strains that are hyper transmissible, hyper resistant, have enhanced capacity to form biofilm, and have reduced susceptibility to chlorhexidine in order that transmission of these key virulence factors can be targeted, and so new detection systems for these dangerous pathogens and associated virulence factors can be constructed and matched with therapeutics such as vaccines, 5) linkage of 1-5 to predictive models that identify patients at increased risk of development of one or more healthcare-associated infections (HCAIs), 6) linkage of 1-6 to an automated system for tracking patients that have suffered from one or more HCAIs following surgery, and 7) packaging 1-6 into a state-of-the art surveillance system designed to maximally attenuate ESKAPE bacterial transmission and subsequent HCAI development, a multi-level, laboratory-based surveillance system that impacts patients during the entire perioperative period, and can be extended beyond to acute care settings to long term care facilities and to the food and pharmaceutical industries. An innovative software platform and implementation plan supports 1-7.

In an initial aspect, the present invention targets three different potential reservoirs of pathogens (patients, provider hands, and the surrounding environment) and downstream catheter care to attenuate bacterial contamination of high-risk intravascular devices. The present invention involves systematic-phenotypic-genomic surveillance systems that characterize the epidemiology of bacterial transmission events occurring in operating room environments. The systems prevent bacterial transfer and subsequent 30-day postoperative healthcare-associated infection (HCAI) development. Systematic-phenotypic and genomic processes are tied together to identify bacterial transmission events, to characterize the epidemiology of those transmission events [(reservoir of origin, mode of transmission, portal(s) of entry, portal(s) of exit, and pathogen strain characteristics, to identify hyper transmissible, hyper virulent, and hyper resistant bacterial pathogens (clinically relevant pathogens)], and to identify and develop rapid, point-of-care diagnostics for genetic traits that characterize clinically relevant pathogens. Ultimately, this information generates feedback loops for infection control healthcare workers that support dynamic, proactive improvements in basic preventive measures and generates novel diagnostic tools and molecular markers for infection that can lead to the development of new disinfection and/or therapeutic agents (antibiotics). Further, the diagnostics developed as a result of this system employment can, in parallel, be matched the therapeutics derived from the same system. Feedback is stratified according to systematic-phenotypic and genomic outputs.

Systematic-phenotypic processing involves the identification of epidemiologically-related bacterial transmission events and key reservoirs of origin, portals of entry/exit, modes of transmission, and pathogen strain characteristics that are remarkable at the phenotypic level. This information supports regular feedback, daily updates, which is primarily utilized to continually inform improvements in hand hygiene, patient decolonization, and environmental cleaning at group levels and to guide preoperative, customized antibiotic therapy.

In one aspect, the systematic-phenotypic process in parallel guides a subset of bacterial isolates down a genomic processing pathway that via the confirmation of clonal relationships, identifies with greater specificity reservoirs of origin, specific modes of transmission, and specific pathogen strain characteristics. As these relationships are established at the nucleotide level, this information is used to support focused improvements at the individual level (specific provider hands, specific pieces of equipment, etc.), with feedback generated bimonthly.

In one aspect, the present invention provides specific output, which differentiates the present clonal processing from other approaches (such as systematic-phenotypic processing loop), comprising identification of established bacterial reservoirs within an environment (institutional reservoirs), such as for example an operating room that infected patients, providers, and equipment over days to months. In addition, the genomic processing leads to the development of rapid, point-of-care diagnostics. These products can provide feedback within an hour, and as such, can be applied to dissect the cause of a reservoir identified by the genomic pathway by large scale sampling of multiple reservoirs in the process of patient care related to environment being tested. These diagnostics can also be used to measure the fidelity of applied preventive measures real-time in order to insure that they are effective.

In brief, the system components can comprise 1) a proprietary database that identifies patients at risk for infection development (pre-procedure analysis of patient Key Performance Indicator (KPI), 2) a lab process for systematic collection and analysis of known intraoperative bacterial reservoirs that leverages the platform of temporal association (previously validated) and utilizes a proprietary analytical pathway to interpret bacterial isolates ultimately yielding the identification of epidemiologically-related and clonal transmission events, 3) at least one connection through SQL, Application Program Interface (API), or Health Level Seven International (HL7) to an Electronic Medical Records (EMR) system, 4) a proprietary, post-procedure analysis of KPI to identify those patients who do develop infections, 5) a proprietary process that integrates processes 1-4 to a) examine the relationship of bacterial transmission to infection, b) to characterize the epidemiology of bacterial transmission, and c) to identify clinically relevant bacterial strains that escape current measures in that they are hyper transmissible, hyper virulent, and/or hyper resistant to antibiotics, 6) a systematic, genomic process that identifies single nucleotide variants and associated functional consequences in clinically relevant bacteria in order to identify new traits conveying antibiotic resistance and new molecules for antibiotic and disinfection therapy, and 7) a process that leads to construction of customized, rapid, point-of-care diagnostics for the most dangerous bacteria in a given operating room environment.

Infection risk information can comprise information relating to a bacterial strain, as well as information relating to the patient. Information relating to a bacterial strain can include, for example, phenotype, genetic sequence data, genotype, virulence, antibiotic resistance, and transmissibility. Patient information can include information that is normally part of an electronic medical record, or otherwise capable of identifying a particular patient or connecting the patient to a particular sample or event. Examples include a patient identification number, a barcode, name, date of birth, and the like. Patient information may also include medical history, and information relating to pre-operative, intra-operative, and post-operative arenas that the patient came into contact with, as well as healthcare providers with which the patient came into contact. In an exemplary embodiment, patient demographic information is provided to an information system associated with the pre-operative arena. Patients can be identified, for example through use of a web-based application, in the preoperative period.

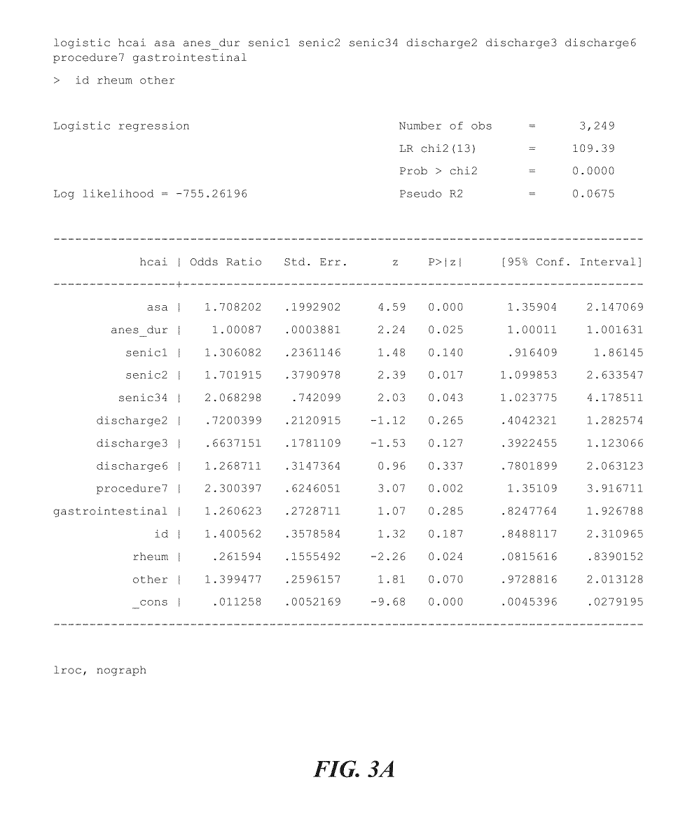

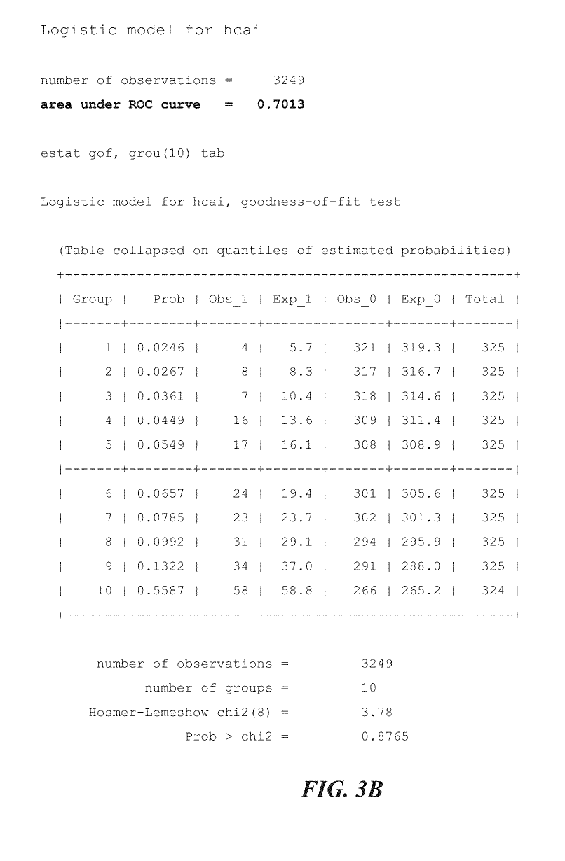

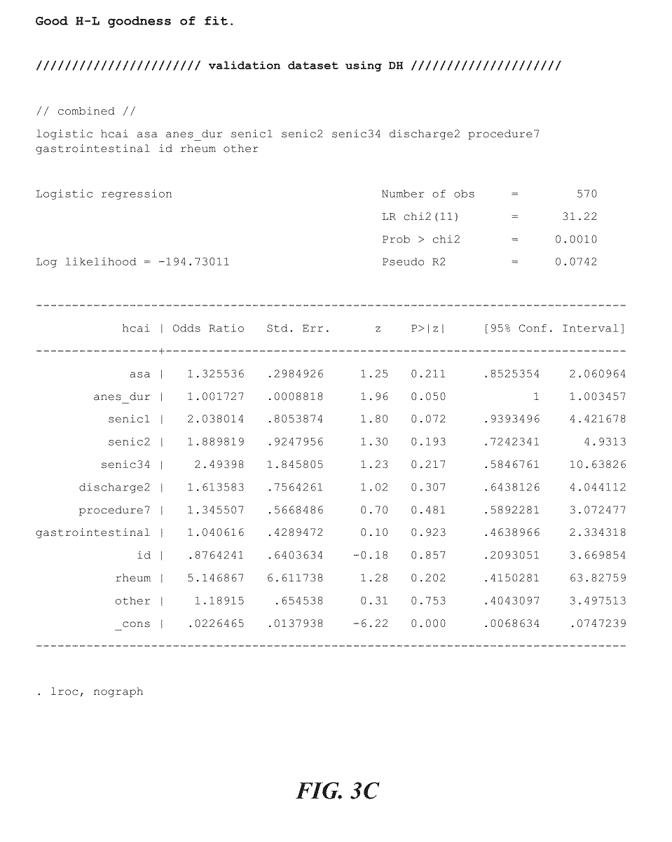

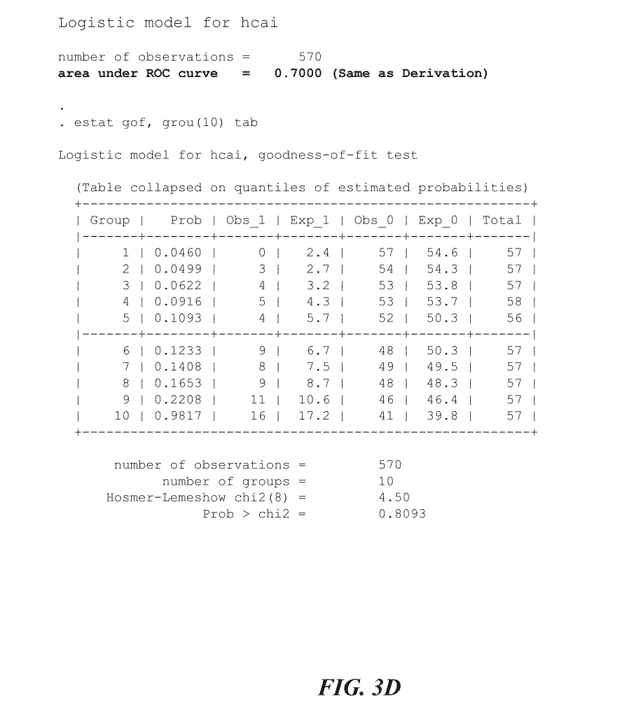

In one aspect, the present methods involve screening patients for development of infection or for high risk for development of infection. In a further aspect, the screening comprises processing infection risk information. The infection risk information used in the processing can be associated with a particular patient, with one or more pre-, intra-, or post-operative arenas, or one or more healthcare providers, or combinations thereof. In a further aspect, information systems are provided in one or more of these units, wherein the each information system automatically process patient demographic information based on predictive modeling to identify those who are high risk and need to be screened. In one embodiment, the processing comprises a preoperative scoring system to identify the patients at risk for infection development. In an exemplary embodiment, the processing is performed using an automated program as shown in FIG. 3A-D.

Screening may further comprise a laboratory-based surveillance system. Laboratory-based surveillance may be conducted by analyzing samples obtained from patients, healthcare providers, and/or arenas that are preoperative, intra-operative, and/or post-operative. In one aspect, pre-operative samples can include nasopharyngeal, axillary, and inguinal swabs obtained from patients. In an exemplary embodiment, for patients with planned colorectal and/or urological/gynecological surgery (higher risk for Enterococcal and/or gram negative transmission), a rectal swab can be obtained. In a further aspect, intra-operative samples can include samples taken from representatives of the anesthesia work area environment (including equipment, air), samples taken from patient skin sites strongly correlated with surgical site infection development, and samples taken from provider hands. Samples can be collected throughout patient care, and may be time-stamped. In an exemplary embodiment, the samples are collected using swabs, and each swab receives a unique barcode that is then linked to the patient encounter number in the electronic medical record which is also linked to all patient, provider, and procedural demographic information as well as the development of postoperative infections and outcomes such as hospital stay duration and mortality. In another aspect, postoperative samples can include those collected from key reservoirs, such as healthcare provider hands, environmental sites, wounds, and air/equipment. The samples can be collected from a period spanning before patient entry to 48 hours postoperatively.

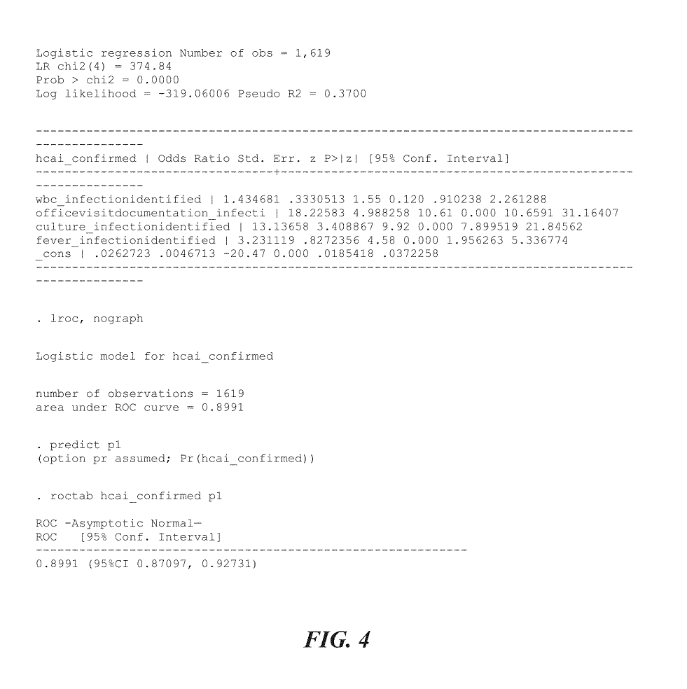

In another aspect, the present invention comprises a surveillance process for detecting patients who develop infections and/or require the acquisition of patient cultures for workup of infection. In an exemplary embodiment, the detection is performed according to the automated process as shown in FIG. 4.

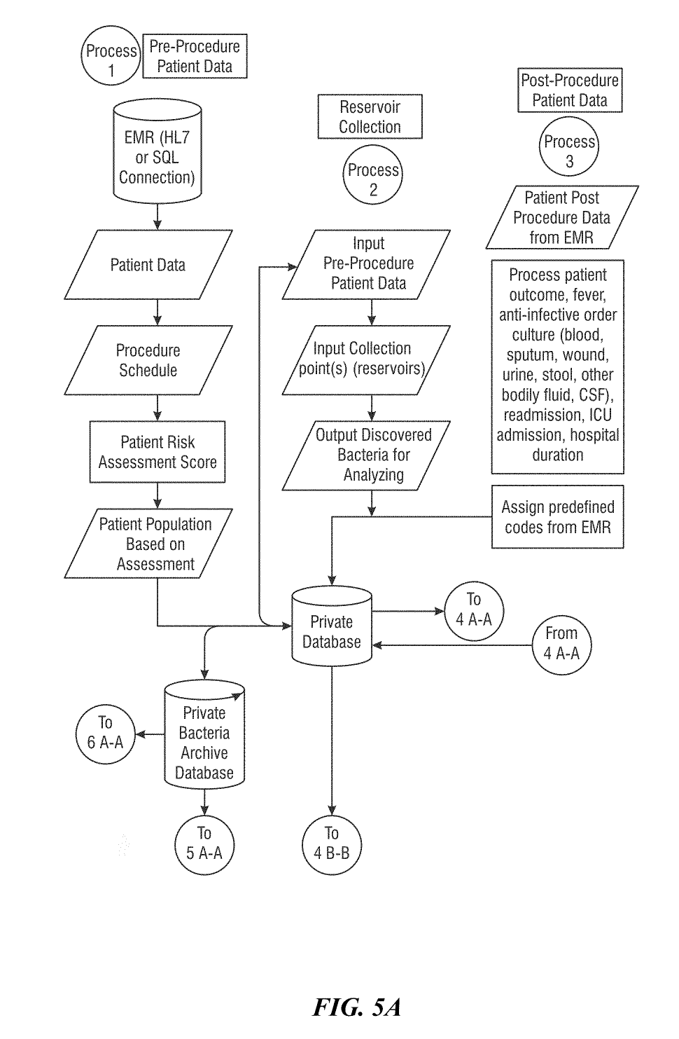

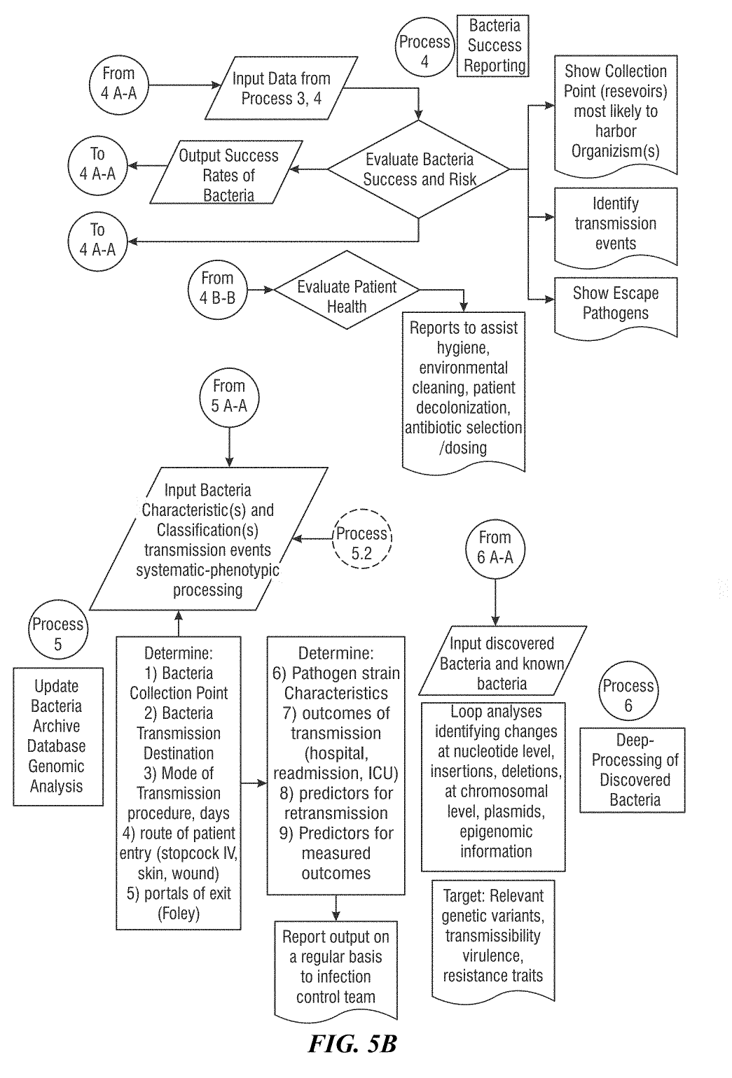

In another aspect, the present invention comprises a computational system for identifying patients that become infected, bacterial transmission events, the epidemiology of bacterial transmission events, whether bacterial transmission events are linked to infection development, and identifies bacterial isolates that are hyper transmissible, hyper virulent, and/or hyper resistant to antibiotic therapy (clinically relevant bacterial pathogens). In an exemplary embodiment, as depicted in FIGS. 5A-B, the system integrates the predictive modeling for identification of patients at risk of developing postoperative infections (FIG. 3), laboratory testing, and surveillance process for detecting patients who develop infections and/or require the acquisition of patient cultures for workup of infection. This involves use of a laboratory guidance system to generate a 23-digit BugID. The BugID then guides a substrate of isolates down a refined pathway of genomic analysis.

In a further aspect, the methods also comprise evaluation of clinically relevant bacterial isolates to identify structural variants, including single nucleotide variants, insertions, and deletions from known strains and the functional consequences of those structural variations, including amino acid changes, impact on 3-dimensional protein structure, and the impact of those conformational changes on antibiotic binding sites. This process also identifies variants that are unique to clinically relevant pathogens, serving as the substrate for both the development of rapid, point-of-care diagnostics and for the development of new therapeutic/disinfection agents.

In a further aspect, the methods further comprise continual automated predictive modeling for identifying patients that become infected, bacterial transmission events, the epidemiology of bacterial transmission events, whether bacterial transmission events are linked to infection development, and identifies bacterial isolates that are hyper transmissible, hyper virulent, and/or hyper resistant to antibiotic therapy (clinically relevant bacterial pathogens).

In a further aspect, the methods comprise evaluating clinically relevant bacterial isolate. The evaluation comprises producing bacterial isolates from environmental or patient samples; determining the bacterial class of one or more bacteria derived from said bacterial isolate; determining the biotype of bacteria derived from said bacterial isolate; and comparing two or more bacteria derived from different bacterial isolates to assess epidemiological relation. In an exemplary embodiment, bacterial isolates are produced by the process of ESwab collection comprising rolling over surface area 10 times, transport to laboratory, vortexed 15 seconds, collection buffer spun at 1,500 rpm to generate a pellet, pellet re suspended in 100 uL tryptic soy broth, plated on to conventional 5% sheep's blood agar (zig zag with 90-degree rotation to cover all quadrants) and incubated at 35 degrees Celsius for up to 48 hours, typically overnight. If plated to large 5% sheep's blood agar plates, 1 mL placed using same technique. In another exemplary embodiment, the determination of bacterial class via use of the automated laboratory guidance system comprises gross morphology and simple rapid tests that through an automated decision making algorithm are used to identify the class of pathogen that is used as the first digit of the 23-digit BugID and guides subsequent isolate handling on the pathway of identification of ESKAPE ERTEs and genomic-refined ESKAPE ERTEs.

### Bacterial Archive

In a further embodiment, the present invention includes bacterial archives wherein bacterial isolates are linked by barcode to the methods and systems as described herein, and wherein the archive is searchable by outcome of interest. The bacterial isolates comprising the archive can be obtained from patients, from healthcare providers, and/or from pre-, intra-, and post-operative arenas. In one aspect, the archive can comprise a multiplicity of sample storage devices, wherein said sample storage devices are configured to store bacterial, and wherein said sample storage devices are operatively connected to an interface for communicating with a computing device to provide information relating to a bacterial isolate contained in the sample storage device. The interface for communicating with a computing device can be, for example, a barcode, a QR code, a SD interface, or a USB interface. The bacterial samples can comprise intact bacteria, including preserved bacterial cells, or bacterial genetic material.

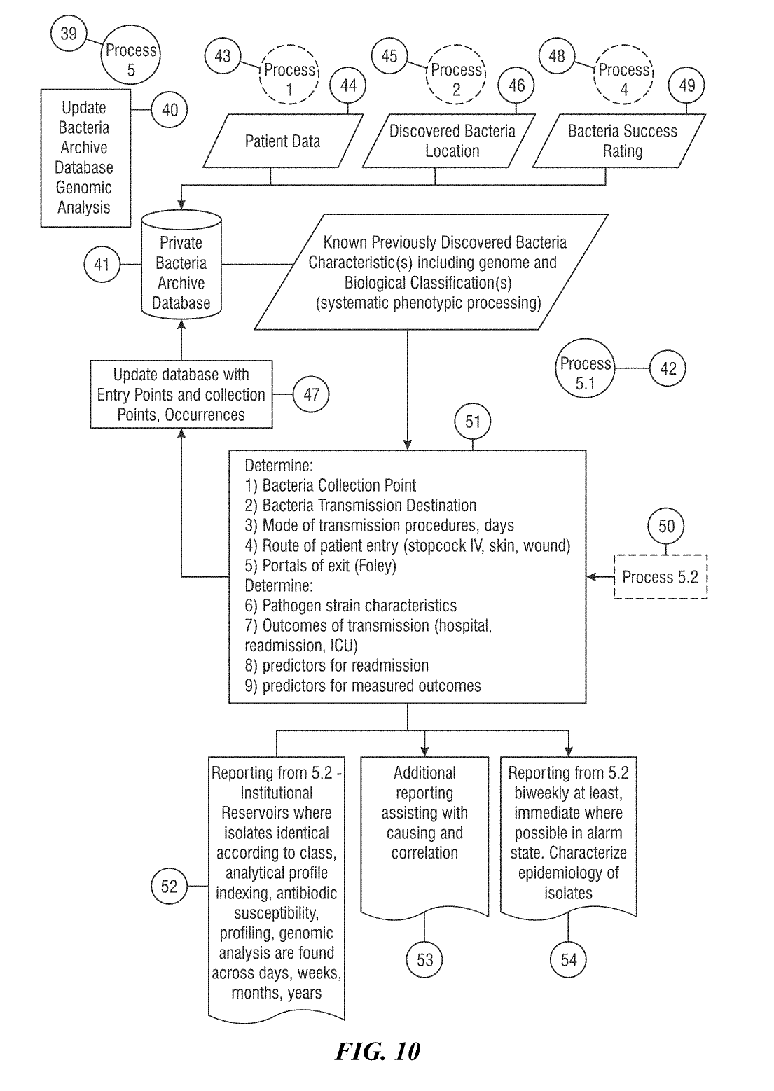

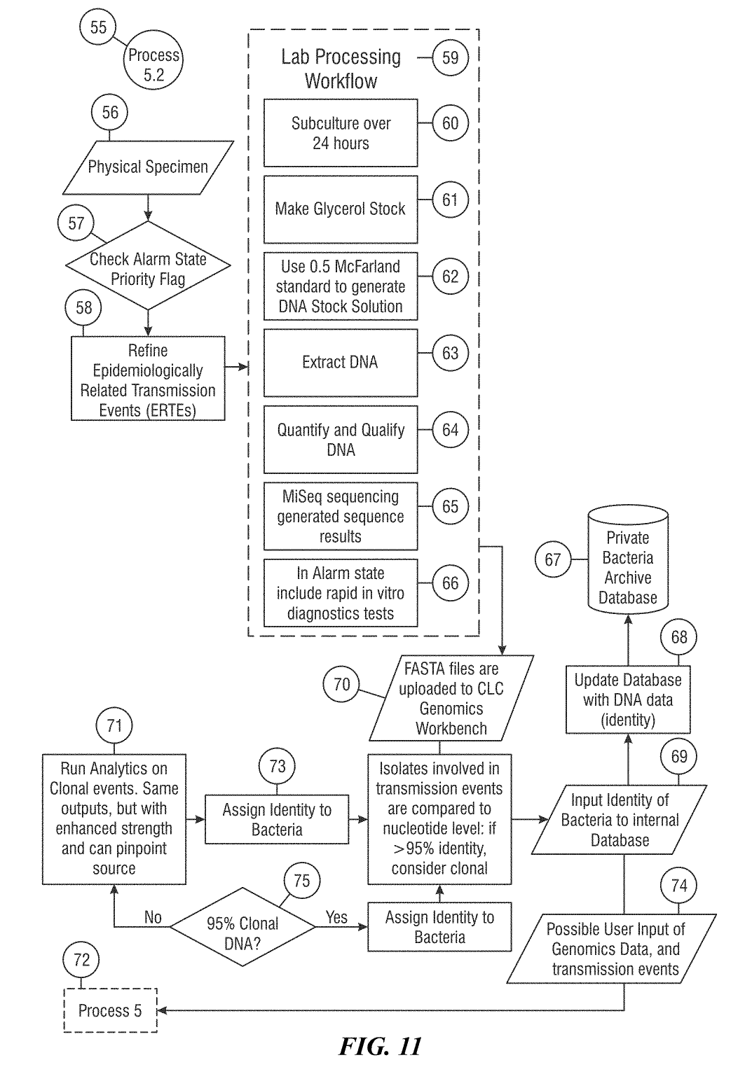

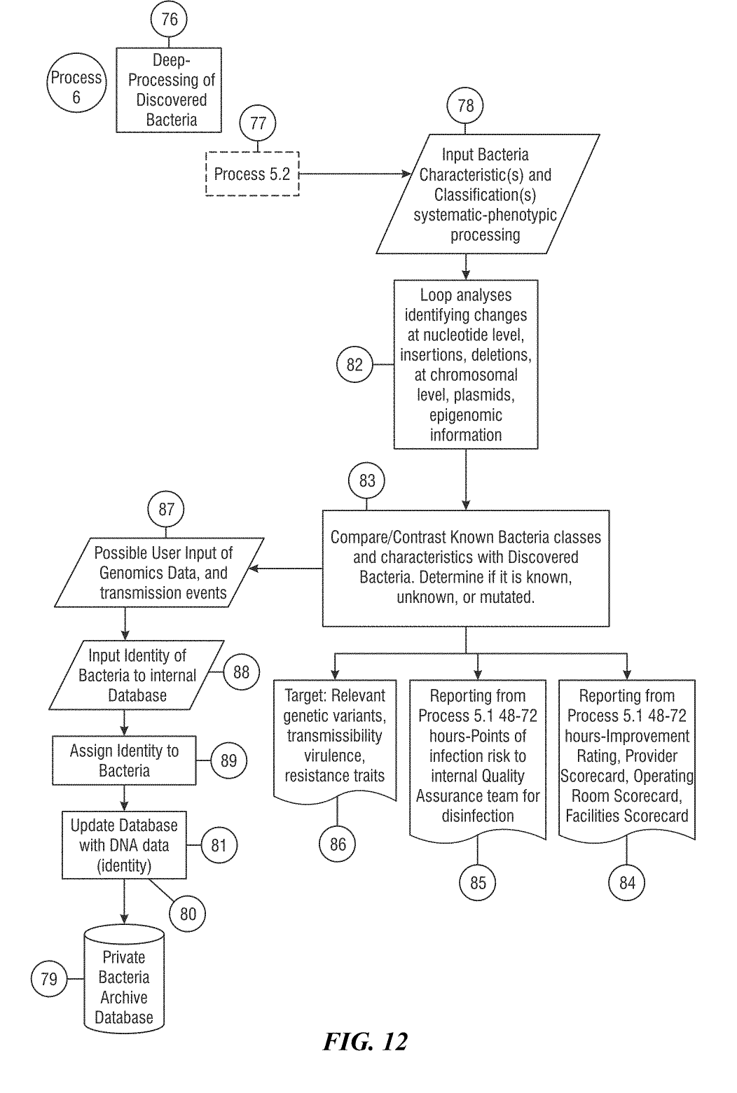

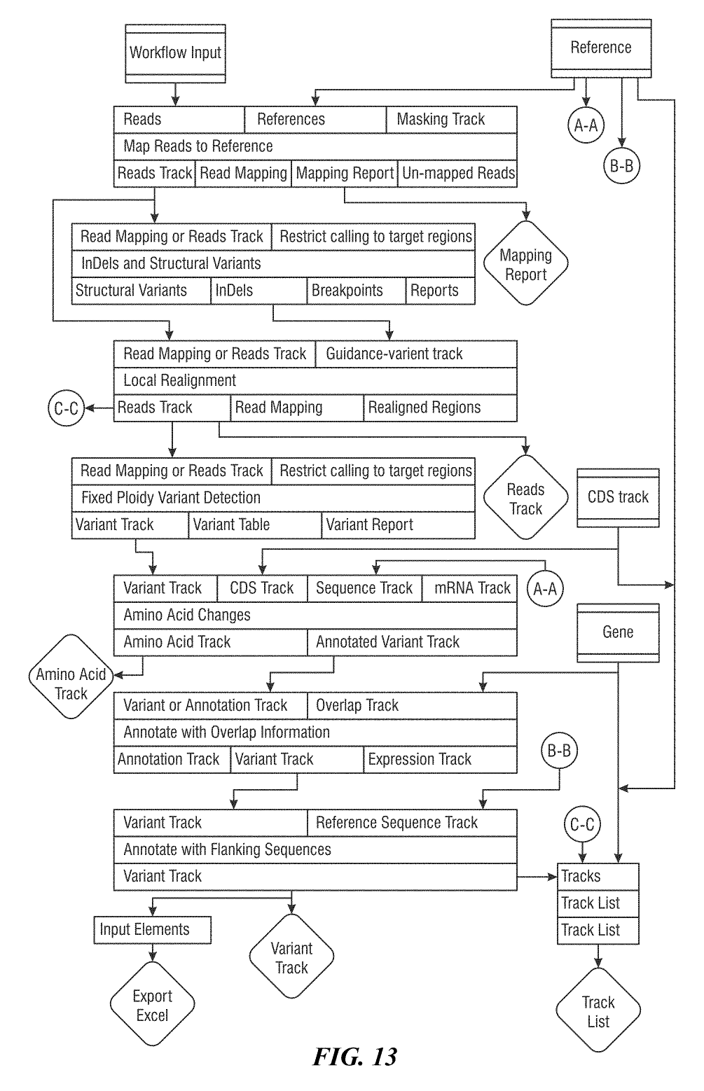

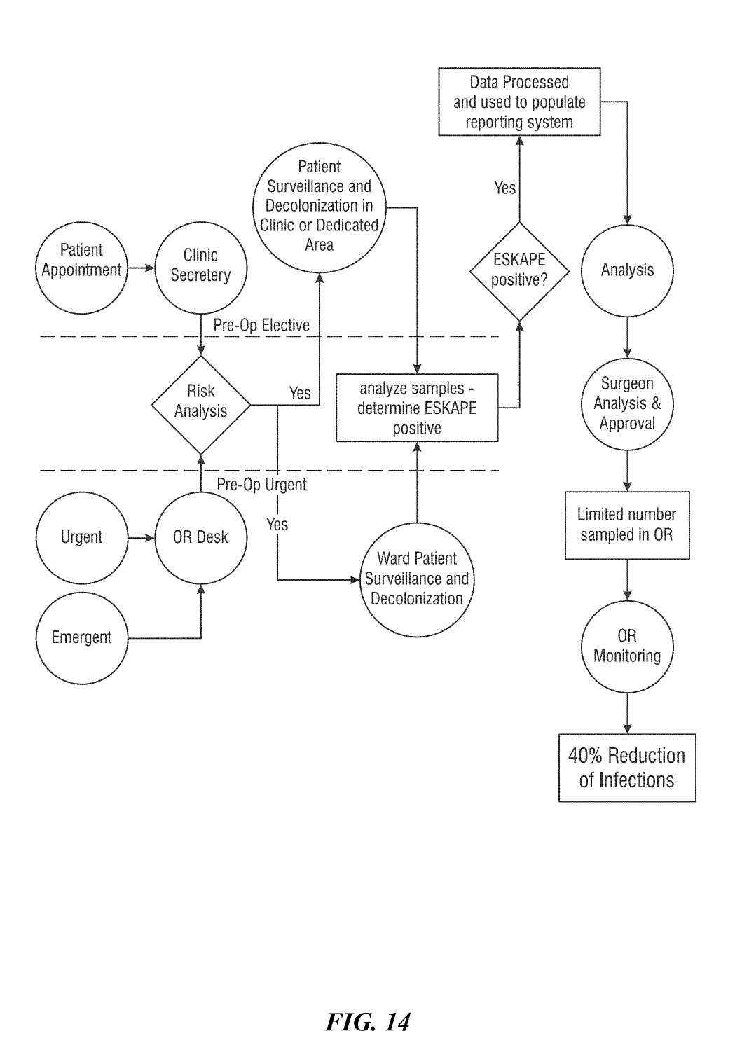

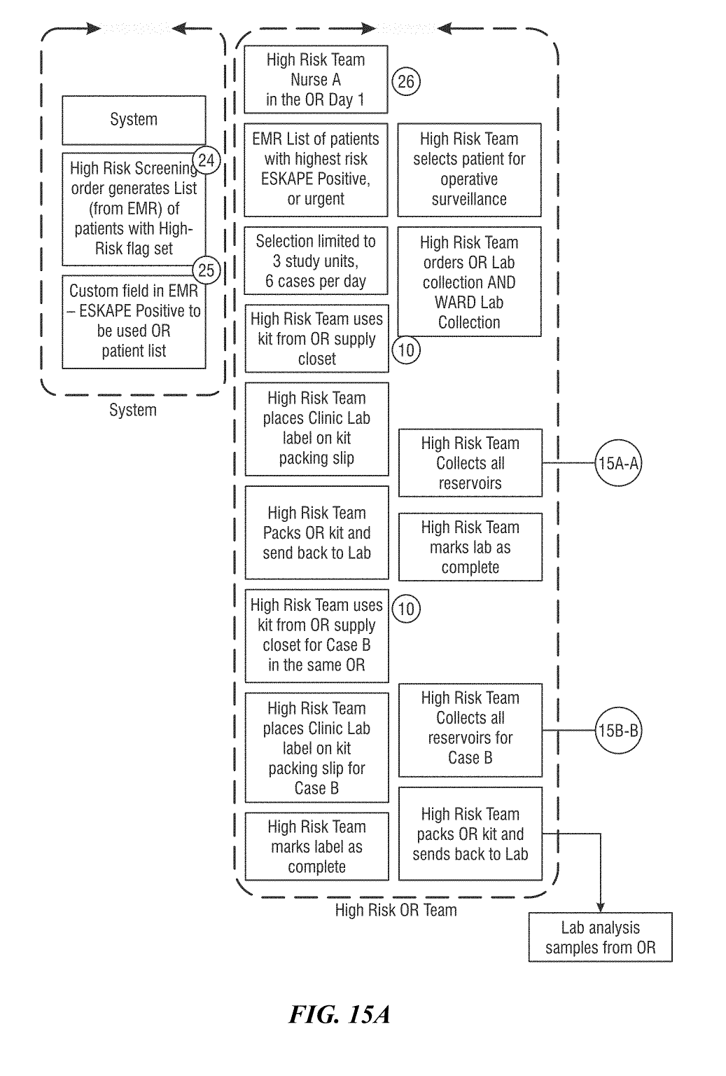

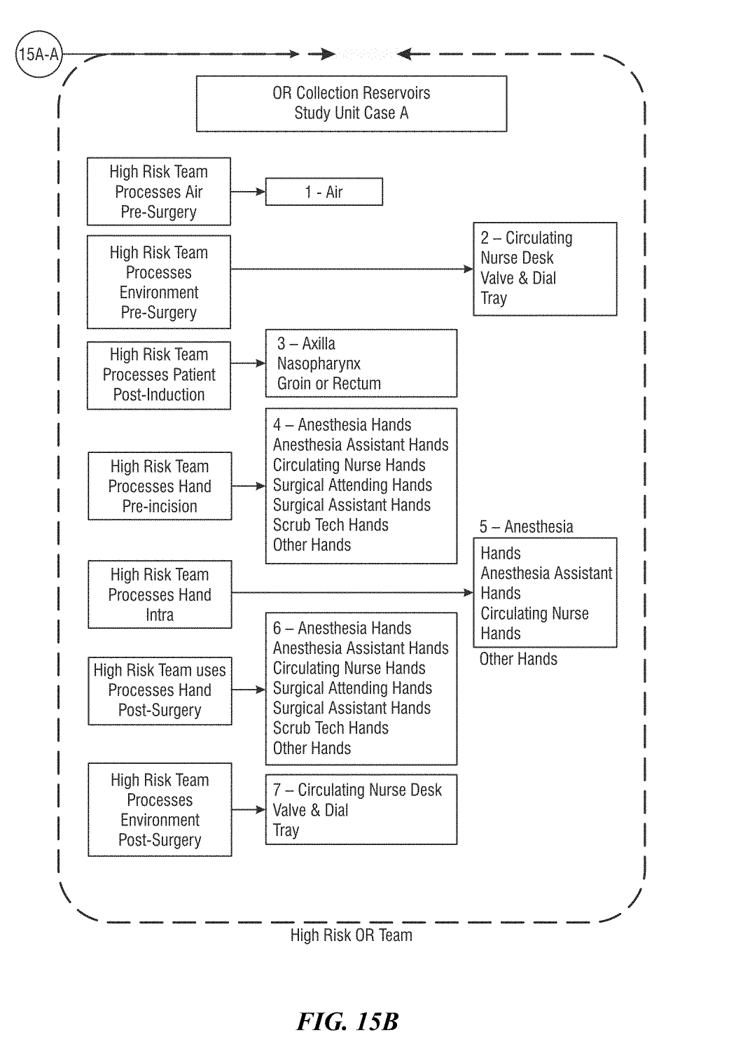

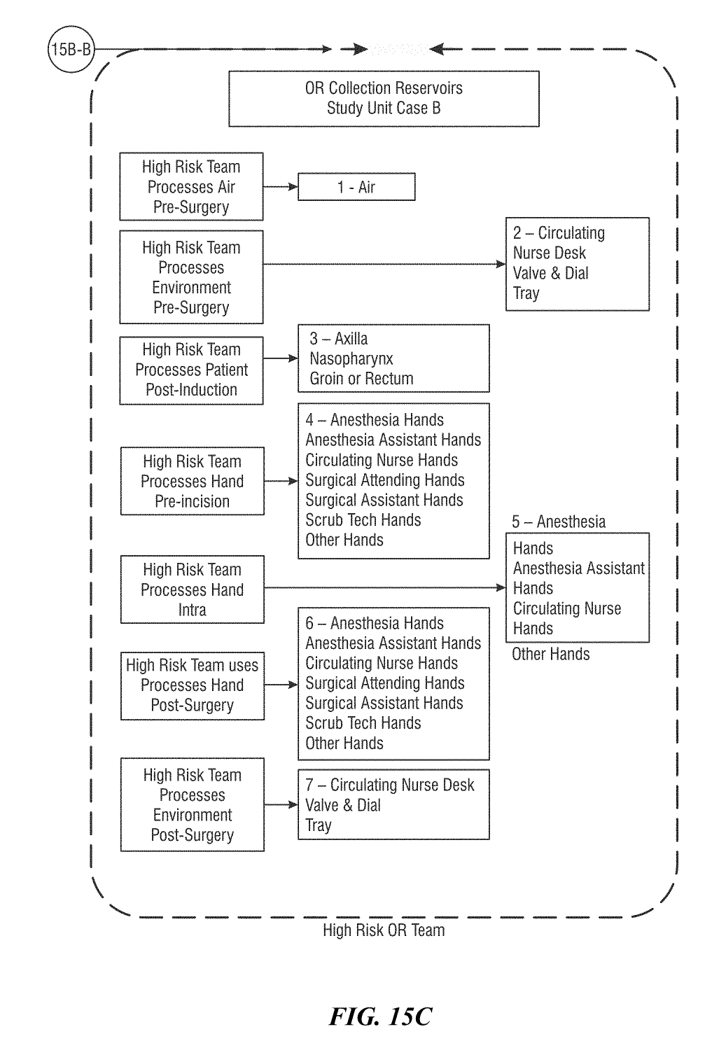

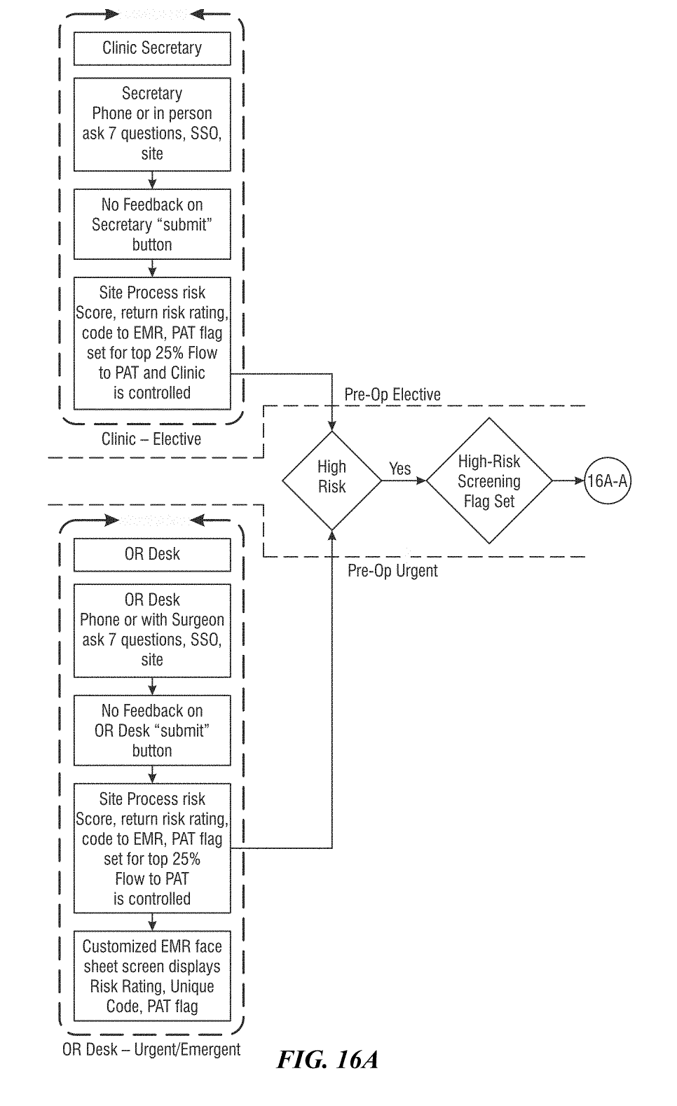

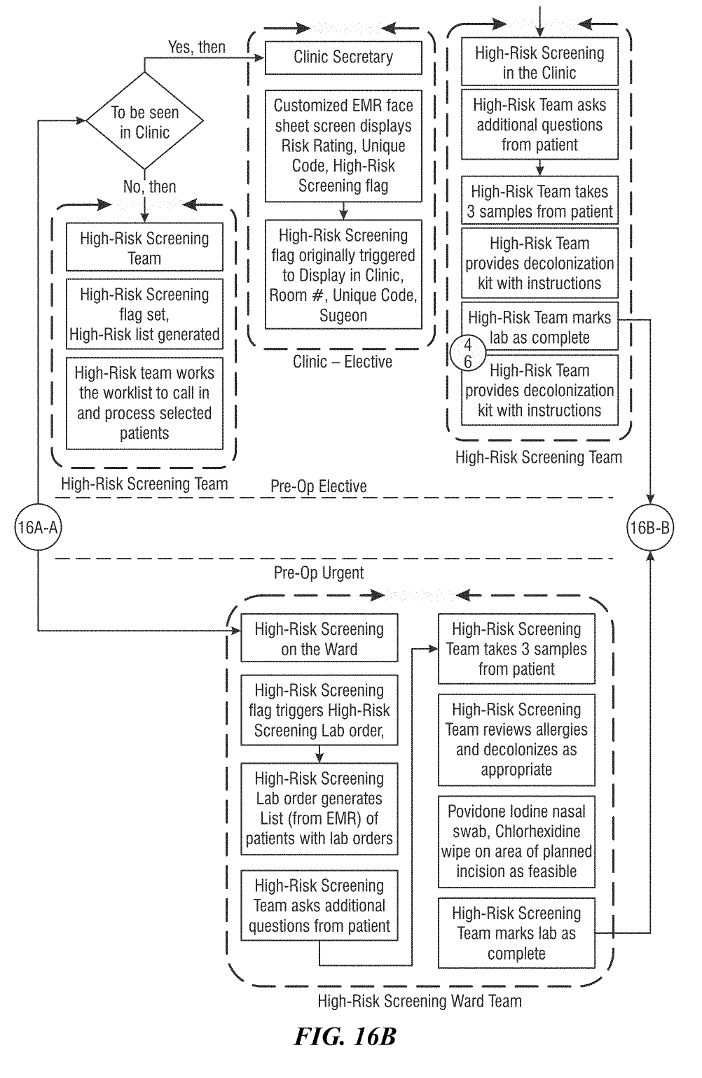

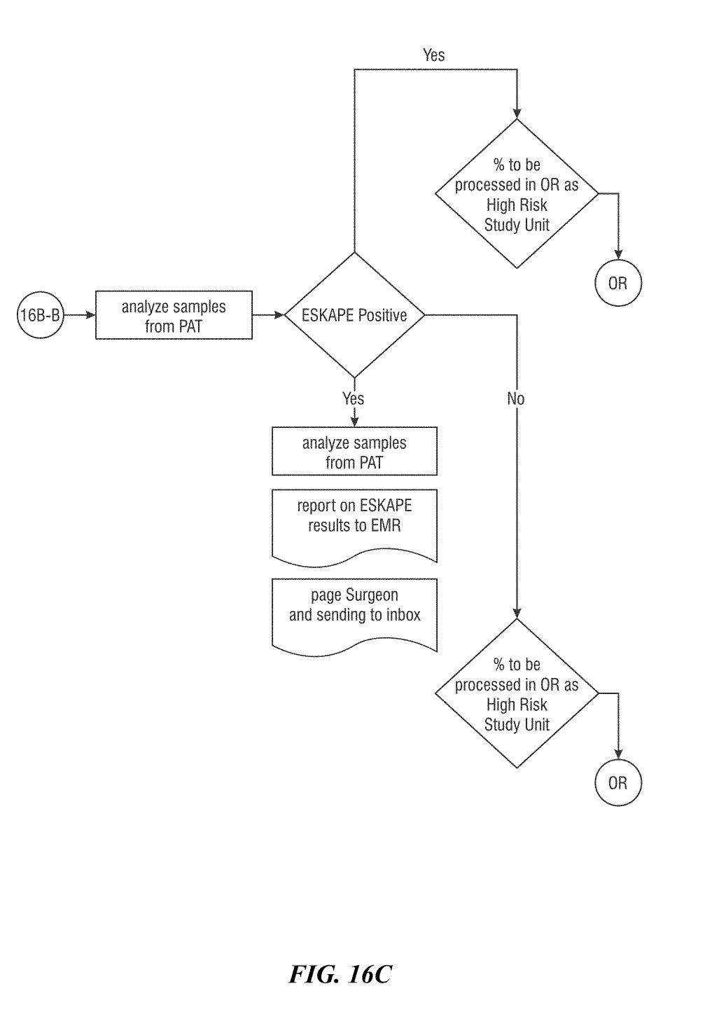

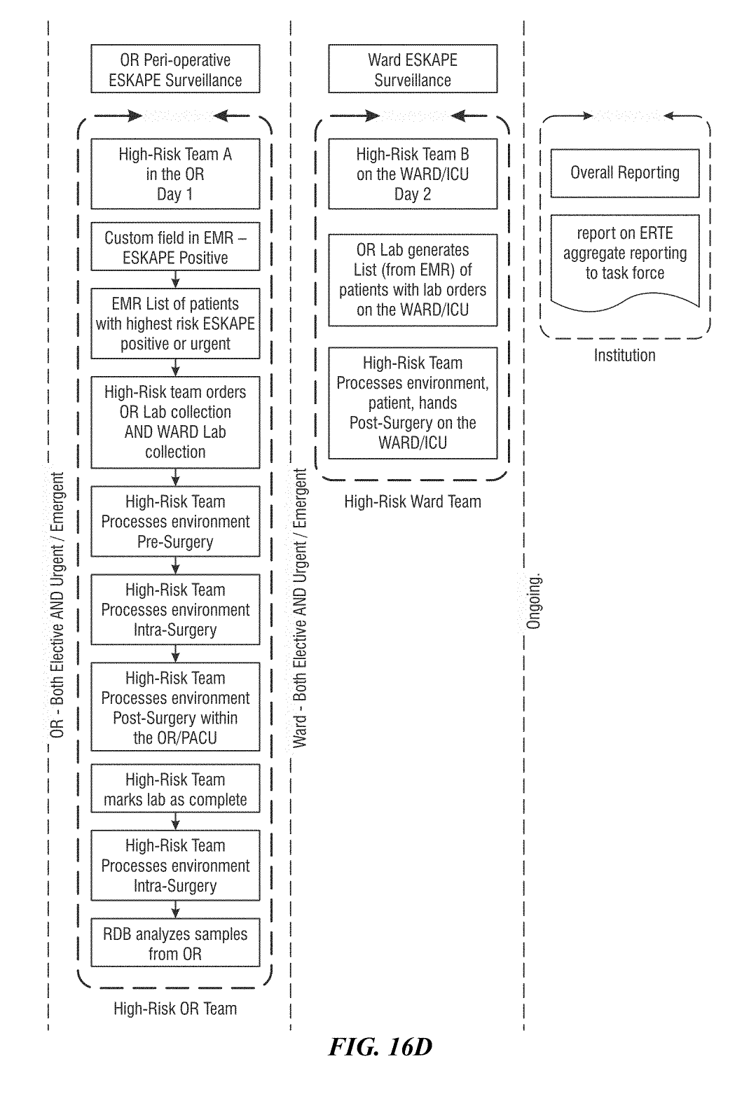

As shown in FIG. 10, the first step of Process 5 involves identifying epidemiologically-related ESKAPE transmission events. This requires automated bacterial processing through a laboratory guidance system that generates a unique 23 digit code (BugID). The first code is generated by the guidance system that guides the user down a step-wise, automated approach to determine whether a reservoir isolate is probable S. aureus, Enterococcus, Pseudomonas, other gram negative, coagulase negative Staphylococcus, micrococcus, streptococcus, corynbacterium, or bacillus. The laboratory guidance system directs the user based on rapid testing results ultimately arriving at initial pathogen classification. If classified as 1-4, the pathogens are frozen and archived for subsequent analysis. If classified as 5-9, the pathogens are frozen and archived for subsequent analysis only if they involve the stopcock reservoir (due to continuity with the patient intravascular space, these pathogens are more likely to cause infection). Pathogens archived for subsequent analysis are analyzed by the software program to evaluate a possible transmission series, where an isolate with the same initial classification is found in an operative case pair (case and case to follow). These isolates are considered possibly epidemiologically-related and are flagged, guiding the user, for analytical profile indexing and a specific order of antibiotic susceptibility testing that leads to a 22 digit number. This now 23 digit number, the BugID, guides isolates down the pathway for next generation sequencing and whole genome analysis (see FIG. 13). Only those pathogens involving an isolate with the same BugID present in more than one location in an observational unit (case pair) are considered for genomic analysis. This output refines epidemiologically-related mapping (used for group level improvement strategies) to yield focused strategies.

This process is first used to compare and report epidemiologically-related transmission events stratified by time, by class of surgery, by type of ESKAPE pathogen, by overall transmission, by biofilm formation, by chlorhexidine susceptibility, by stopcock involvement, and by hyper transmissible and antibiotic biomarkers. Epidemiology-related transmission events (ERTEs) and associated epidemiological factors are mapped to specific operating room environments and text files provided to the end user by clicking on the flagged operating room that specifically recommend up-to-date, evidence-based improvements driven by ESKAPE transmission data. The text files drive improvements today to deal with the occurrence of events, while predictive analytics are used to provide associated co-variates for the flagged epidemiological variables (specific provider and patient groups, environmental cleaning of certain areas, etc.) in order to prevent future recurrence.

The next level of analysis occurs at the nucleotide level in order to identify clonal events defined when 2 or more pathogens that are epidemiologically-related also have whole genome similarity based on multi loci sequence testing, >98% nucleotide identity to a common reference, and in some cases, belong to the same nucleotide variant cluster. Clonal transmission events are used to refined ERTE maps to increase the sensitivity and specificity of event detection, and to identify and characterize the source of institutional reservoirs defined by the presence of the same organism for greater than 7 days in a given environment.

The next level of analysis in the process is to take data collected from the previous processes, all linked to a unique barcode, demographic ID, and case-log-ID, to identify and to summarize nucleotide variants, insertions, and deletions that associate with bacterial success identified in Processes 1-4, which serves as the substrate for development of rapid, nucleic acid-based diagnostics.

The next level of analysis is to use genomic analysis to characterize resistance traits associated with hyper transmissible strains and to map the transmission of those traits, thereby providing actionable targets for specific attenuation of resistance traits, such as extended-spectrum beta lactamase resistance traits that prolong and increase the severity of infections when they develop.

The next level of analysis is to identify BugIDs that are institution specific. This creates an institutional finger print in order that infections can be assigned to the institution in case of a match, or not assigned in the case of a mismatch. This impacts infection reporting and Medicare reimbursement. Further, mismatches between resistance traits (genomic) and antibiotic susceptibility are detected by the system. These mismatches identify strains that are developing emerging mechanisms of resistance. The software system automatically stratifies commonly employed antibiotics according to the frequency of mismatch detect and considers this data input for prophylactic, empiric, and therapeutic antibiotic recommendations in order to augment antibiotic stewardship in the post antibiotic era. In the case where there are 3 antibiotics that could be used, the system recommends the one with the fewest number of institution-specific isolates with emerging resistance patterns.

The next level of analysis is map the odds of operating rooms according to ESKAPE transmission. This is meant to guide operating room throughout, matching immunocompromised patients at increased risk of infection (predictive modeling via the software) with the lowest risk environments. A match is differentially defined.

The next level of analysis is to continually report operating room risk and associated factors on monitors displayed in the operating room and to an infection control perioperative team. The monitors display today's comparative risk of infection and reasons for that risk based on ERTE and genomic-refined ERTEs for ESKAPE transmission. The monitors display the action plan that guides usual, daily processes (terminal and routine cleaning, hand hygiene, and vascular care), empowers surgeons, anesthesiologists, technologists, and nurses to highlight and address deficits before and during the time out (to enhance skin decontamination in the case of flags for reduced susceptibility to chlorhexidine which would be addressed with longer and dual skin antisepsis). The infection control perioperative team is empowered by the system to oversee these processes, as the system automatically records and tracks the fidelity of interventions employed, continually measuring the impact within and between ESKAPE genera, in order that fatigue can be addressed early and mitigated, and in the case that the intervention is exacerbating either transmission or resistance within another class (butterfly effect, swarm theory), the intervention can be stopped.

The next level of analysis is to provide a perioperative catalog of daily ESKAPE exposure. As operating rooms are randomly selected during the process, the data generated by the surveillance system and hosted by the software platform represents the overall arena. With the prevalence of ESKAPE transmission thereby reflective of the true prevalence, the true performance parameters of the system can be routinely measured and optimized. By tracking ESKAPE exposure effectively, surgeons can by using a search engine in the system better select antibiotics when addressing a patient that may be infected without a known organism (ask the system what the exposure in the operating room where the patient received surgery had been over the preceding 5 days at the phenotypic and genomic levels and of those pathogens, what are the most transmitted pathogens, those that they patient was most likely to have been exposed to, susceptible to at the population phenotypic and genomic levels), is infected with a known pathogen (ask the system to use the laboratory guidance process to identify a BugID for the pathogen and use the BugID to characterize and report the population epidemiology of the BugID at the phenotypic and genomic levels, especially important with hetero resistant isolates where a sensitive isolate can mask the presence of a resistant isolate, for methicillin-resistant S. aureus, and thereby lead to undertreatment), or when selecting prophylactic antibiotics before surgery (using the same logic, is the patient high risk for infection, is the patient ESKAPE positive, if ESKAPE positive, what is the BugID and associated epidemiology, including phenotypic and genomic resistance patterns), and ask the system to continually address antibiotic stewardship by flagging and targeting high risk patients with preventative approaches such as nasal mupirocin use in order to avoid widespread use associated with increasing resistance, by tracking emerging resistance among institution-specific isolates, by comparing institutions regarding these isolates stratified across antibiotics to identify successes and failures of antibiotic prescribing patterns, and by using the information to guide selection of antibiotic therapy.

### Collection Kits

In a preferred embodiment, the methods and systems can comprise a collection kit. Preferred collection kits include OR PathTrac Kits. OR PathTrac kits are proven, unique bacterial collection system that works in conjunction with the OR PathTrac Laboratory guidance and reporting software platforms.

Proven bacterial reservoirs of the intraoperative environment can be systematically sampled throughout the duration of a surgical case pair (2 cases observed in series). Preferably, each case has a dedicated surveillance kit. Up to 35 sites can be surveyed per OR case via use of the kits, and up to 32 reservoirs can be assigned. One or more reservoirs, preferably at least two reservoirs, more preferably three reservoirs, are not assigned but kept at the discretion of the end user for the provision of extra supplies. Extra supplies can be used to fill gaps between surveillance points. The kits can be layered to organize the end user, with each layer containing simplistic diagrams that guide the sampling process.

Preferably reservoirs can comprise the following: AHO1, AHA01, SgH01, SgHAss01, CNH01, SgTechH01, OH01, AnesHAttIO, AnesAssistHIO, CNHIO, OHIO, AnesAttHE, AnesAssHE, SgHE SgAssHE CNHE, SgTechHE, OHE, LE, AnesVD01, CND01, InstrumTray01, AnesVDIO, CNDIO, AnesVDE, CNDE, InstruTrayE, PN1, PA1, Pg1, PR1, PNE, PAE, with to be determined samples (N=3).

Preferably each of reservoir receives two unique barcodes. One barcode specific to the reservoir, which is preferably consistent throughout all kits. For example, AH01=xxxxxxx, CNH01=xxxxxx, where xxxxxx is the barcode consistent among the kits. This code is recognized by the OR PathTrac Laboratory guidance system to identify the reservoir. The second barcode provides a unique ID for each reservoir unit that links the sampling unit to the kit, the case pair assigned to the kit during sampling, all bacterial specimens subsequently derived from the reservoir sample unit, and all patient, provider, procedural demographics for the demographic observational unit (case pair).

The ORPathTrac Kit reservoir units are strategically sampled over time, from case start to case end. This reservoir ordering system leverages the platform of temporal association which allows the identification of bacterial transmission event series. A transmission event is defined by one or more pathogens present at an intraoperative or case end sample that were not present at case start. Each transmitted isolate is compared via a systematic-phenotypic-genotypic analytical process to identify the reservoir of origin, mode of transmission (between/within case), portal of entry, transmission locations, and strain characteristics (antibiotic resistance and traits/transmissibility/reduced chlorhexidine susceptibility/biofilm formation/contamination of intravascular devices/and links to infection). Phenotypic analysis yields epidemiologically-related transmission stories (ERTES). ERTES can be refined by genomic analysis. Both ERTES and genomic-refined ERTES can be analyzed via complex statistical and computational analyses by a reporting platform to generate meaningful reports that can guide global and focused, respectively, improvements in antibiotic selection (empiric, prophylactic, and therapeutic antibiotic selection, antibiotic stewardship, patient decolonization, hand hygiene, environmental cleaning, catheter care, operating room throughput).

Preferably the ORPathTrac Kits are IATA certified for transport of biological materials, category B, such that the institution simply obtains the kit, uses the contents, places the contents back in the box in any order, closes the box, places it in a provided over pack, and ships the samples to a laboratory. In some embodiments, the site can transport the kits to their own laboratories for internal processing.

### Laboratory Guidance System

A laboratory, which can be on-site or off-site, can receives the collection kit, which can be scanned for identification. Subsequently the kit can be propagated and processed. An exemplary embodiment of kit reception, propagation, and processing is provided below. It should be understood that this embodiment is exemplary and can be modified in many ways.

### Step 1: Receiving the Kit.

The sample kit ID can be scanned. This ID leads to the display of all kit contents in three layers.

A. Reservoir hand samples (N=18)

B. Lumen sample: N=1

C. Environmental samples: N=8

D. Patient samples: N=5

E. Extra samples: N=0-3 depending on the user

Preferably, the kit and the contents are marked received.

### Step 2: Propagating the Kit

Each layer can be assigned a specific label printing pattern that guides sample propagation to the correct number and type of agar plates.
A=Sample spun, pellet re-suspended in 1 mL with 1004 of the sample subsequently diluted 1:2, 1:5, and 1:10 and 100 μL of each diluent transferred to standard sized blood agar plates.
B=Entire 1 mL volume of the sample collection fluid transferred to a large blood agar plate.
C=Sample spun, pellet re-suspended, transferred to standard blood agar plates.
D=Same as C
E=For the hand sample, same as A, for other samples, same as C.

The labels, with the unique sample barcode (assigned to the sample during kit preparation) and associated directions for propagation as above, can be affixed to the samples.

A user can propagate the samples as directed, place the appropriate label on the appropriate medium. The sample can be transferred to the incubating location. Each plate now has a barcode that links subsequent isolates to the demographic unit and all attached information as previously described. The status, triggered by printing labels for each sampling unit, is now incubating, ready for basic processing. This status generates a list for the user that populates at 24 hours from the label printing. The user knows what samples are ready for basic processing. Preferably, a search function can be provided that allows the user to search for any unique ID to determine the status and all associated factors.

### Step 3: Basic Processing

This step can guide a user through a process to begin construction of unique BugIDs (biomarkers) for each unique colony that grows from a reservoir unit. This BugID informs the reporting platform. The technologist works the list of ready for basic processing and obtains the samples from the incubating location. The samples can be scanned in, and this automatically changes the status of the samples to processing for basic analysis.

### Basic Analysis Process A:

a. Record growth yes, no
b. Count and record the number of colony forming units on each plate. This can be done manually (conventional technique) and via an automated process where a picture of the plate is generated, and via USB connection and processing, the colonies (dots) are counted and reported to a lab guidance software.
c. Characterize/observe the morphology of the colonies (size as small, medium, large, and very large, color as white, grey, black, yellow, golden, hemolysis as gamma, beta, alpha, presence of dimpling yes/no) and identify all unique colonies. This will be done manually and by computer processing as above. Each unique colony receives a unique barcode linked to the demographic code, reservoir code, and kit ID.
d. One of each unique colony from a plate are streaked to blood agar plates and incubated. The system populates a list for the user 24 hours from the start of processing for basic analysis status.

### Basic Analysis Process B:

a. The user works the samples ready for basic analysis list.
b. Checks for growth: records yes/no
c. Gram stain:
   1. Gram positive, gram negative, bacilli, coccobacilli, diplococci, lancet-shaped, pairs
d. If gram positive, perform catalase and oxidase test, record as positive or negative.
e. If catalase positive, oxidase negative, do coagulase test.
f. If catalase positive, oxidase negative, coagulase positive, do ornithine test.
g. If catalase positive, oxidase negative, coagulase positive, ornithine negative, grow on mannitol salt agar plate.
h. If gram negative, do lactose fermentation test.
i. If lactose fermentation is negative, do oxidase test
j. If lactose fermentation negative and oxidase positive, do indole test.
k. If indole positive, do urease test.

Software logic basic analysis guidance: The software automatically processes the above information to generate the first code of the BugID.

Code 1: probable S. aureus: gram positive cocci, catalase positive, oxidase negative, coagulase positive, ornithine negative.

Code 2: Probable Enterococcus: gram positive cocci, gamma hemolysis, catalase negative.

Code 3: Probable pseudomonas: Gram negative bacillus, nonfermenting, oxidase positive, indole negative.

Code 4: Other GN: based on gram stain, not otherwise specified as above. This could be Acinetobacter, klebsiella, Enterobacter, E. coli, citrobacter, serratia, other.

Code 5: Probable coagulase negative staphylococcus. Gram positive cocci, catalase negative, oxidase negative, coagulase negative

Code 6: Probable micrococcus: gram positive cocci, catalase positive, oxidase positive

Code 7: Probable Coryn-gram positive rods

Code 8: Strep: Catalase negative. If gamma hemolysis, lancet-shaped diplococci, otherwise alpha or beta hemolysis

Code 9: Probable bacillus: large, amorphous, grey colonies

The software can generate the probable assignment and asks the user to confirm based on colony size, morphology, and presence/absence of hemolysis. If the user objects and reassigns, the code, this is tracked.

Software logic to freeze: The program will always assign codes 1-4 for preparation of glycerol stock, and 5-9 if the isolate is in or matches the lumen sample. This logic populates a freeze worklist with each sample to be frozen with a check box. When the box is checked, a label will be printed with the unique sample barcode to be affixed (as directed on the label) to tryptic soy broth, 5 mL, for inoculation with the sample and overnight growth. The status will automatically change to processing for freezing. This will populate a work list 12 hours from the status change to processing for freezing. The sample list will be worked by the user. When the box is checked, a unique freezer location will be assigned to be affixed to the glycerol stock, cryocentrifuge tube as directed. This freezer location will be linked to all sample information.

Logic can proceed with additional sampling. Preferably, software processes the demographic units to identify where in the case of codes 1-3, the probable isolate is present in more than one location. If codes 4-9, the sample must be in the stopcock to move on to additional processing, and there cannot be a gram stain mismatch or >2 colony size/morphology mismatches to move on.

Codes 1-3: >1 location, possible epidemiologically-related transmission event (PERTE), to API/KB testing involving 22 specific biochemical tests. If 4-9, in stopcock, and without gram stain or >2 other mismatches in colony size, morphology, hemolysis, then off to API/KB. The results populate a list of samples with status now ready for KB/API. The user works the list. When the box is checked by the sample, 10 labels are printed, 5 for KB, 5 for API, with each label containing the unique barcode.

Epidemiologically-related transmission events (ERTEs): Basic processing, API and KB codes yield a unique BugID number. Where present in >1 site in a demographic unit, this is an ERTE. This populates a list of samples with status ready for genomic analysis. The samples when checked trigger label printing for DNA extraction, 10 per sample.

DNA extraction complete: marked in software, assigned a new freezer location, status ready for sequencing.

The DNA can be sequenced, linked to the original code, multi-loci sequence testing and single nucleotide variant analysis used to re-align transmission links, genomic-refined ERTEs. Done automatically by the software. This process uses the MLST code and also a variant cluster analysis that yields unique codes.

### Logic for Transmission Event Identification Processing

According to an aspect of the invention, a match is differentially defined. Level 1 processing is used to identify reservoir transmission events. A match is defined by class of

ESKAPE pathogen, where 2 or more reservoirs are the same class of pathogen (0=Enterococcus, 1=S. aureus, 2=Klebsiella, 3=Acinetobacter, 4=Pseudomonas, 5=Enterobacter)-1 digit BugID. Level 2 processing is used to stratify reservoir transmission events into those that are epidemiologically-related (ERTEs). This requires the addition of a unique 22-digit number generated by biological testing, now level 2 BugID with 23 digits. Level 3 processing is used to refine ERTEs via use of genomic analysis where a 6-digit number, now the BugID (complete) has 29 digits.

Preferred logic can comprise the following:
T01D: Never an event
T01APL: Never an event
AH01: If matches with T01D, T01A
RH01: If matches with T01D, T01A
CRNA01: If matches with T01D, T01A
OH01: If matches with T01D, T01A
PN1: If matches with T01D, T01A, AH01, RH01, CRNAH01, OH01
PA1: If matches with T01D, T01A, AH01, RH01, CRNAH01, OH01
NOL1: If matches with T01D, T01A, PN1, PA1
AHE1: If different from AH01, If matches with T01D, T01A, RH01, CRNAH01, OH01, PN1, PA1
   If matches with NOL1 as long as NOL1 is different than AH01, RH01, CRNAH01, OH01
RHE1: If different from RH01, If matches with T01D, T01A, AH01, CRNAH01, OH01, PN1, PA1
   If matches with NOL1 as long as NOL1 is different than AH01, RH01, CRNAH01, OH01
CRNAHE1: If different than CRNAH01, If matches with T01D, T01A, AH01, RH01, OH01, PN1, PA1
   If matches with NOL1 as long as NOL1 is different than AH01, RH01, CRNAH01, OH01
OHE1: If different than OH01, if matches with T01D, T01A, AH01, RH01, CRNAH01, PN1, PA1
   If matches with NOL1 as long as NOL1 is different than AH01, RH01, CRNAH01, OH01
TE1D: If different than T01D. If matches with T01A, AH01, RH01, CRNAH01, OH01, PN1, PA1, NOL1, AHE1, RHE1, CRNAHE1, OHE1
TE1A: If different than T01A. If matches with T01D, AH01, RH01, CRNAH01, OH01, PN1, PA1, NOL1, AHE1, RHE1, CRNAHE1, OHE1
LE1: If no match then any positive, can match with T01D, T01A, AH01, RH01, CRNAH01, OH01, PN1, PA1, NOL1, AHE1, RHE1, CRNAHE1, OHE1, TE1D, TE1A
T02D: If different from T01D, If matches with T01A, AH01, RH01, CRNAH01, OH01, PN1, PA1, NOL1, AHE1, RHE1, CRNAHE1, OHE1, TE1D, TE1A, LE1
T02A: If different from T01A, If matches with T01D, AH01, RH01, CRNAH01, OH01, PN1, PA1, NOL1, AHE1, RHE1, CRNAHE1, OHE1, TE1D, TE1A, LE1
AH02: If different from AH01
   If matches with T01D, T01A, RH01, CRNAH01, OH01, PN1, PA1, RHE1, CRNAHE1, OHE1, TE1D, TE1A, LE1
   If matches with NOL1 if NOL1 is different from AH01, RH01, CRNAH01, OH01
   If matches with T02D, T02A
   If matches with AHE1 as long as AHE1 is different from AH01 RH02: If different from RH01
   If matches with T01D, T01A, AH01, CRNAH01, OH01, PN1, PA1, AHE1, CRNAHE1, OHE1, TE1D, TE1A, LE1
   If matches with NOL1 if NOL1 is different from AH01, RH01, CRNAH01, OH01
   If matches with T02D, T02A
   If matches with RHE1 as long as RHE1 is different from RH01
CRNAH02: If different from CRNAH01
   If matches with T01D, T01A, AH01, RH01, OH01, PN1, PA1, AHE1, RHE1, OHE1, TE1D, TE1A, LE1
   If matches with NOL1 if NOL1 is different from AH01, RH01, CRNAH01,

OH01
If matches with T02D, T02A
If matches with CRNAHE1 as long as CRNAHE1 is different from CRNAH01 OH02: If different from OH01
If matches with T01D, T01A, AH01, RH01, CRNAH01, PN1, PA1, AHE1, RHE1, TE1D, TE1A, LE1
If matches with NOL1 if NOL1 is different from AH01, RH01, CRNAH01, OH01
If matches with T02D, T02A
If matches with OHHE1 as long as OHE1 is different from OH01
PN2: If matches with T01D, T01A, AH01, RH01, CRNAH01, OH01, PN1, PA1, AHE1, RHE1, CRNAHE1, OHE1, TE1D, TE1A, LE1
   If matches with NOL1 if NOL1 is different from AH01, RH01, CRNAH01, OH01
   If matches with T02D, T02A, AH02, RH02, CRNAH02, OH02
   If matches with infection culture patient 1
PA2: If matches with T01D, T01A, AH01, RH01, CRNAH01, OH01, PN1, PA1, AHE1, RHE1, CRNAHE1, OHE1, TE1D, TE1A, LE1
   If matches with NOL1 if NOL1 is different from AH01, RH01, CRNAH01, OH01
   If matches with T02D, T02A, AH02, RH02, CRNAH02, OH02
   If matches with infection culture patient 1
NOL2: If matches with T01D, T01A, PN1, PA1, TE1D, TE1A, LE1
   If matches with AHE1, RHE1, CRNAHE1, OHE1 as long as AHE1, RHE1, CRNAHE1, OHE1 are different than AH01, RH01, CRNAH01, OH01
   If matches with T02D, T02A, PN2, PA2
AHE2: If different than AH01
   If matches with T01D, T01A, RH01, CRNAH01, OH01, PN1, PA1, RHE1, CRNAHE1, OHE1, TE1D, TE1A, LE1
   If it matches with AHE1 as long as AHE1 is different than AH01
   If it matches with NOI1 as long as NOL1 is different than AH01, RH01, CRNAH01, OH01
   If it matches with NOL2 as long as NOL2 is different from AH02, RH02, CRNAH02, OH02, AH01, RH01, CRNAH01, OH01
   If it matches with T02D, T02A, RH02, CRNAH02, OH02, PN2, PA2
   If it matches with AH02 as long as AH02 is different than AH01
RHE2: If different than RH01
   If matches with T01D, T01A, AH01, CRNAH01, OH01, PN1, PA1, AHE1, CRNAHE1, OHE1, TE1D, TE1A, LE1
   If it matches with RHE1 as long as RHE1 is different than RH01
   If it matches with NOI1 as long as NOL1 is different than AH01, RH01, CRNAH01, OH01
   If it matches with NOL2 as long as NOL2 is different from AH02, RH02, CRNAH02, OH02, AH01, RH01, CRNAH01, OH01
   If it matches with T02D, T02A, AH02, CRNAH02, OH02, PN2, PA2
   If it matches with RH02 as long as RH02 is different than RH01
CRNAHE2: If different than CRNAH01
   If matches with T01D, T01A, AH01, RH01, OH01, PN1, PA1, AHE1, RHE1, OHE1, TE1D, TE1A, LE1
   If it matches with CRNAHE1 as long as CRNAHE1 is different than CRNAH01
   If it matches with NOI1 as long as NOL1 is different than AH01, RH01, CRNAH01, OH01
   If it matches with NOL2 as long as NOL2 is different from AH02, RH02, CRNAH02, OH02, AH01, RH01, CRNAH01, OH01
   If it matches with T02D, T02A, AH02, RH02, OH02, PN2, PA2
   If it matches with CRNAH02 as long as CRNAH02 is different than CRNAH01
OHE2: If different than OH01
   If matches with T01D, T01A, AH01, RH01, CRNAH01, PN1, PA1, AHE1, RHE1, CRNAHE1, TE1D, TE1A, LE1
   If it matches with OHE1 as long as OHE1 is different than OH01
   If it matches with NOI1 as long as NOL1 is different than AH01, RH01, CRNAH01, OH01
   If it matches with NOL2 as long as NOL2 is different from AH02, RH02, CRNAH02, OH02, AH01, RH01, CRNAH01, OH01
   If it matches with T02D, T02A, AH02, RH02, CRNAH02, PN2, PA2
   If it matches with OH02 as long as OH02 is different than OH01
TE2D: If no match and different from T01D
   If matches with T01A, AH01, RH01, CRNAH01, OH01, PN1, PA1, NOL1, AHE1, RHE1, CRNAHE1, OHE1, TE1A, LE1
   If matches with TE1D as long as TE1D is different than T01D
   If matches with T02A, AH02, RH02, CRNAH02, OH02, PN2, PA2, NOL2, AHE2, RHE2, CRNAHE2, OHE2
   If matches with T02D if T02D is different than T01D
TE2A: If no match and different from T01A
   If matches with T01D, AH01, RH01, CRNAH01, OH01, PN1, PA1, NOL1, AHE1, RHE1, CRNAHE1, OHE1, TE1D, LE1
   If matches with TE1A as long as TE1A is different than T01A
   If matches with T02D, AH02, RH02, CRNAH02, OH02, PN2, PA2, NOL2, AHE2, RHE2, CRNAHE2, OHE2
   If matches with T02A if T02A is different than T01A
LE2: If no match then any positive, can match with T01D, T01A, AH01, RH01, CRNAH01, OH01, PN1, PA1, NOL1, AHE1, RHE1, CRNAHE1, OHE1, TE1D, TE1A, T02D, T02A, AH02, RH02, CRNAH02, OH02, PN2, PA2, NOL2, AHE2, RHE2, CRNAHE2, OHE2, TE2D, TE2A
Patient 1 infection culture: Any positive is a transmission event, can be matched with T01D, T01A, AH01, RH01, CRNAH01, OH01, PN1, PA1, NOL1, AHE1, RHE1, CRNAHE1, OHE1, TE1D, TE1A, LE1
Patient 2 infection culture: Any positive is a transmission event, can be matched with T01D, T01A, AH01, RH01, CRNAH01, OH01, PN1, PA1, NOL1, AHE1, RHE1, CRNAHE1, OHE1, TE1D, TE1A, LE1, T02D, T02A, AH02, RH02, CRNAH02, OH02, PN2, PA2, NOL2, AHE2, RHE2, CRNAHE2, OHE2, TE2D, TE2A, LE2

### ERTE Identification

In a demographic unit (case-pair), there are 2 or more transmission events with the same BugID based on level 2 processing (23 digits).

### Refined ERTE:

In a demographic unit (case-pair), there are 2 or more transmission events with the same BugID based on level 3 processing, 26 digits.

### Mapping Transmission:

### Level 2:

For each ERTE, the transmission series involving isolates with the same 23-digit BugID is ordered by culture acquisition timing from time 0 to case 2 end. The series then receives a unique code constructed based on the ERTE reservoir sequence as follows:

### T01D: 1

### T01A: 2

### AH01: 3

### RH01: 4

### CRNAH01: 5

### OH01: 6

### PN1: 7

### PA1: 8

### NOL1: 9

### AHE1: 10

### RHE1: 11 CRNAHE1: 12

### OHE1: 13

### TE1D: 14

### TE1A: 15

### LE1: 16

Patient 1 infection culture: 17

### T02D: 18

### T02A: 19

### AH02: 20

### RH02: 21

### CRNAH02: 22

### OH02: 23

### PN2:24

### PA2:25

### NOL2: 26

### AHE2: 27

### RHE2: 28

### CRNAHE2: 29

### OHE2: 30

### TE2D: 31

### TE2A: 32

### LE2: 33

Patient 2 infection culture: 34

Now that each ERTE has a transmission code, unique BugIDs are filtered by the number of ERTE events. The top 5 BugIDs for each class of ESKAPE pathogen (1-5) are reported. Each of the top 5 BugIDs are then ordered by unique transmission code. The top 5 (most frequent) transmission codes are then translated for reporting, providing the top 5 patterns of transmission events for the most transmissible BugIDs within an ESKAPE class.

Similarly, to map transmission of hyper transmissible BugIDs that are also resistant, the top 5 BugIDs are ordered by the sum of resistance across the agents tested, and also by resistance for any one agent. BugID transmission codes are then translated to transmission maps.

This same process is used to focus on and map resistant isolates without hyper transmissibility, and for any given virulence factor (chlorhexidine susceptibility, biofilm formation, chlorine resistance).

### Level 3:

The process is the same as level 2, except here the 29-digit BugID is utilized for processing.

### Reporting

Reports can be prepared based on ERTEs and genomic-refined ERTEs including links to postoperative infections. Infections can be tracked over time using OR PathTrac Links to infection (BugID) and institutional tracking via NHSN definitions. Infection incidence can be reported over time with the current incidence displayed in comparison to a target threshold. Overall ERTE and genomic-refined ERTE incidence can also be displayed over time.

In a preferred embodiment, the user selects periods where incidence exceeds benchmark and filters according to the type of surgery, then by the type of ESKAPE (Enterococcus, S. aureus, Klebsiella, Acinetobacter Pseudomonas, and Enterobacter) pathogen. The type of pathogen implicated can direct the user down the analysis pathway for the epidemiology of ESKAPE transmission.

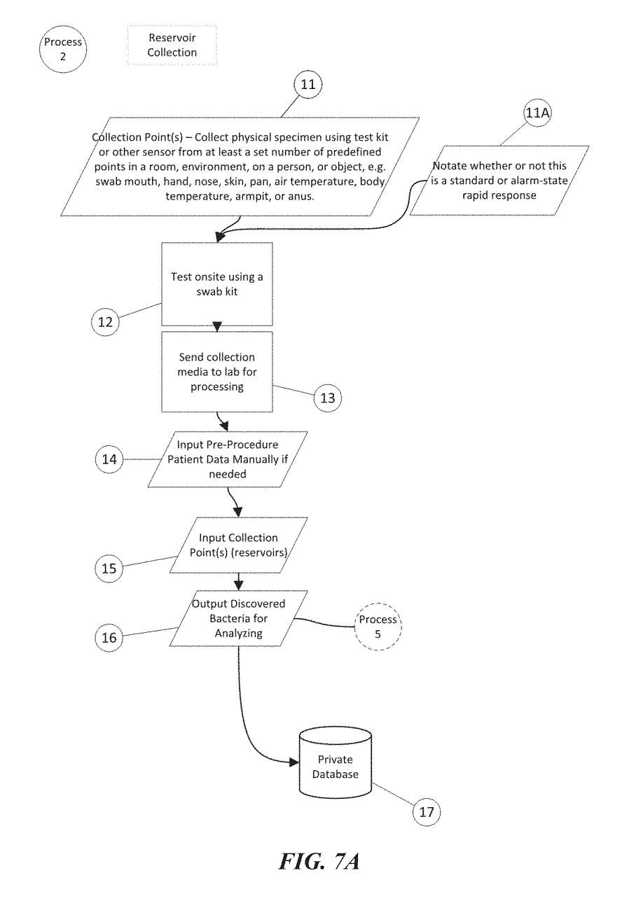

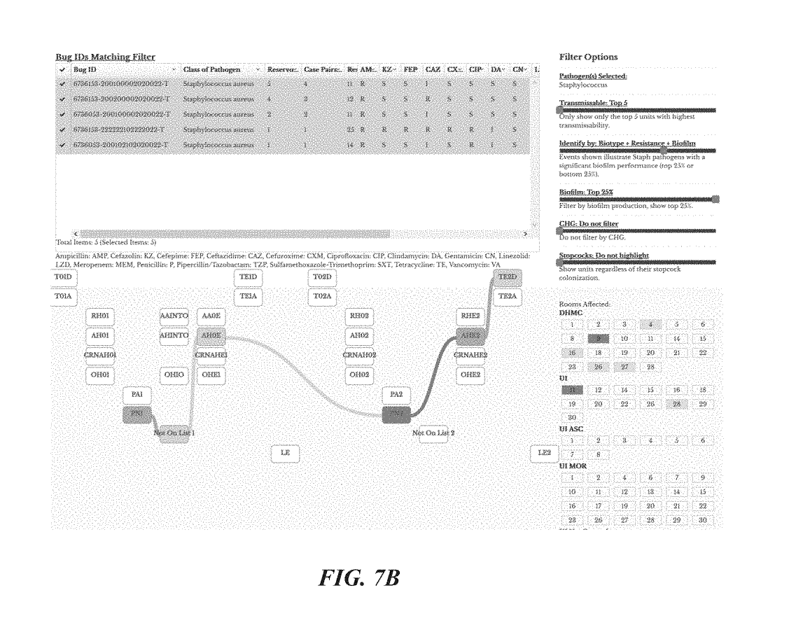

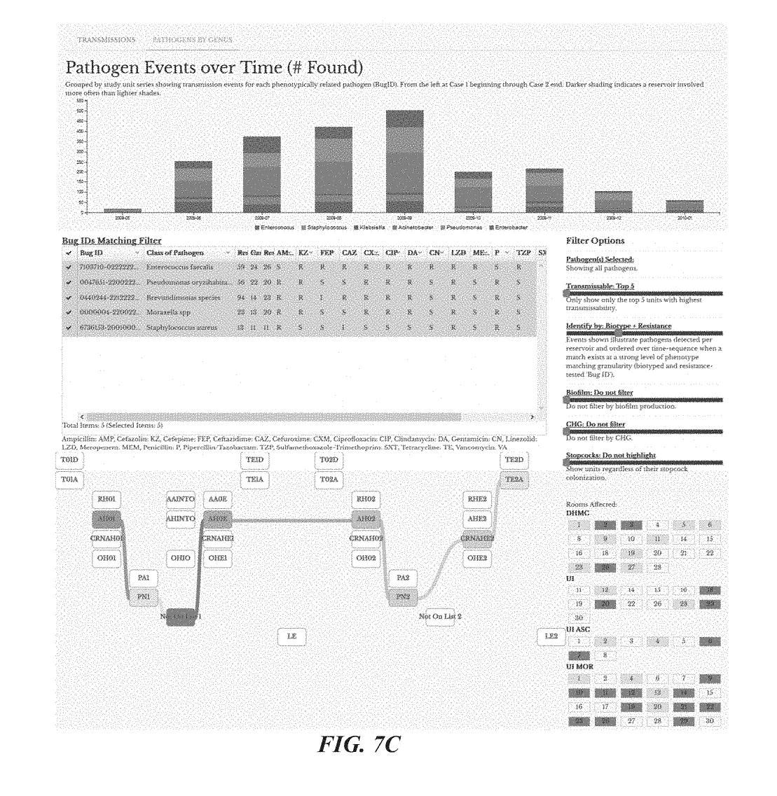

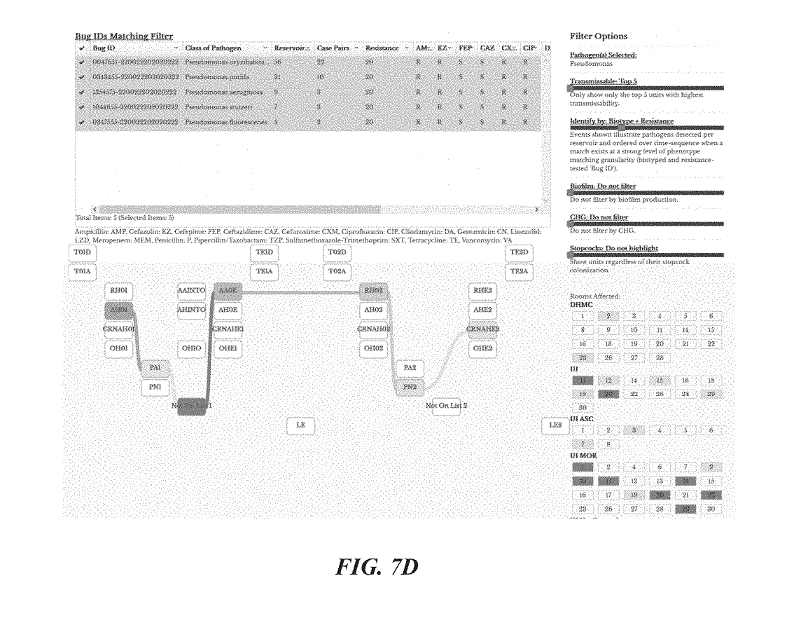

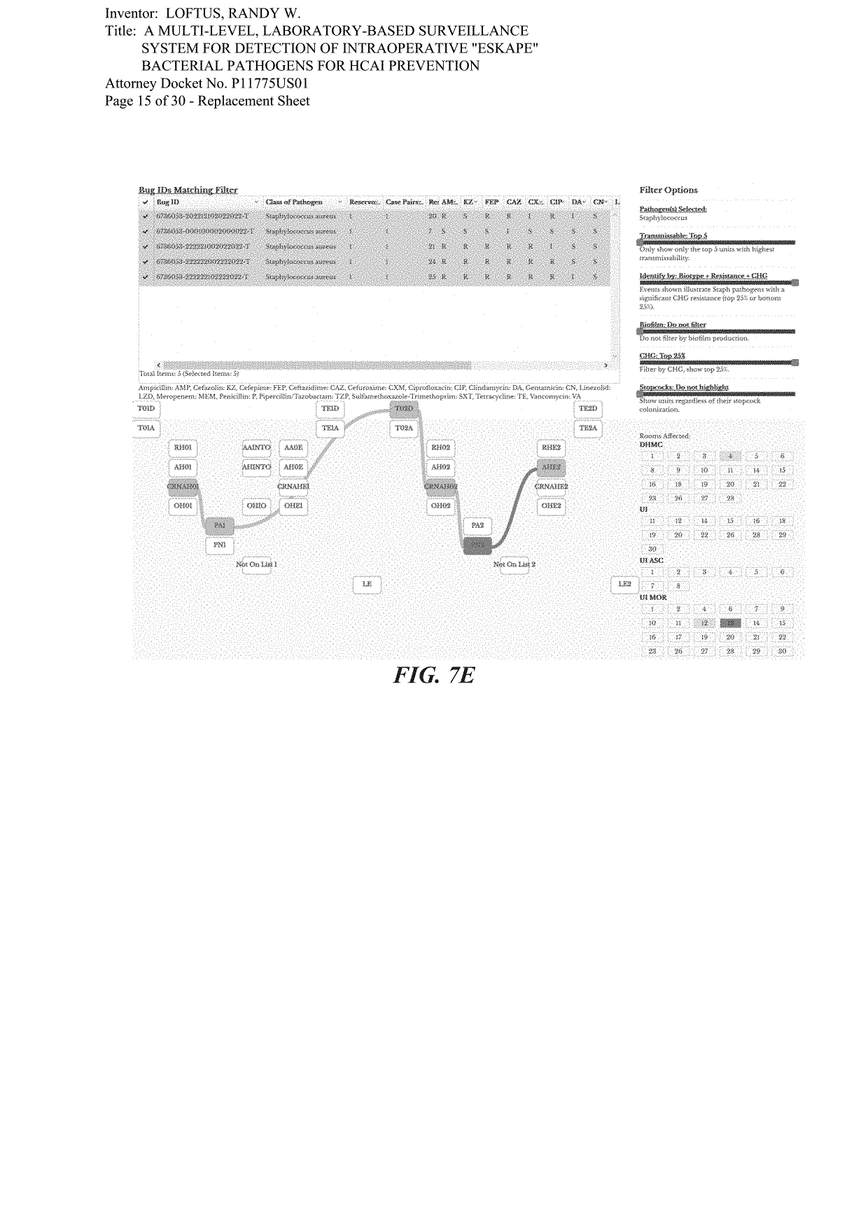

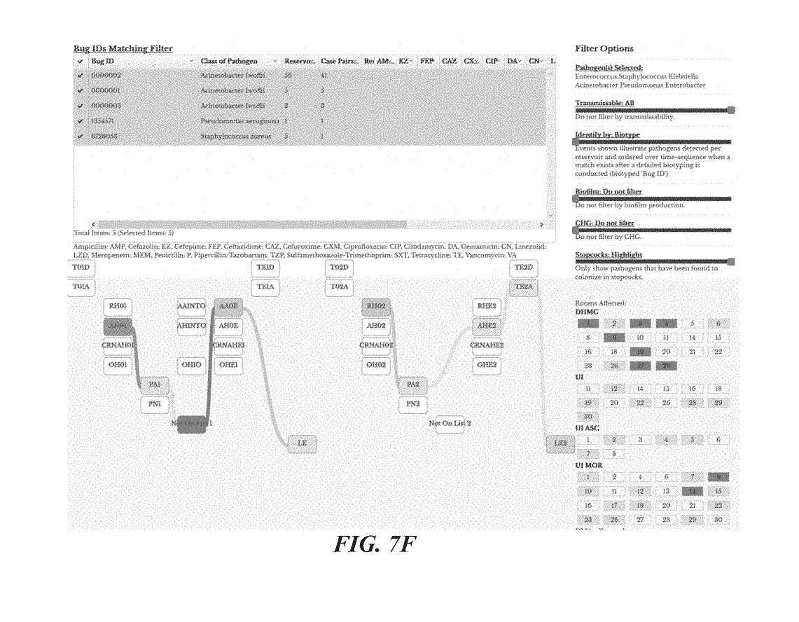

Preferably, the system comprises a software platform that aggregates operating room demographic transmission stories (ERTE and genomic-refined ERTE) over time and identifies the top 3-10, preferably 5, most common transmission routes from reservoir of origin to transmission location, mode of transmission, portal of entry, and links to infection. These patterns can be aggregated and displayed in red arrow diagrams depicting the pathway and identifying targets for improvement based on reservoir-derived algorithms. Images of exemplary red arrow diagrams provided by the system are provided in FIGS. 7B-7F.

The events can be mapped to each OR, updated on a scheduled basis, preferably weekly, more preferably daily, most preferably every 12 hours. As a user selects an OR, the transmission map of the key pathogen affecting the OR is displayed, a text file of recommended actions driven by epidemiological analysis is provided, actionable targets and plan of action today, and predictive analytics and demographic statistics provided for each target to prevent recurrence of the event. This information can be used to optimally attenuate the pathogens driving infections in the postoperative period, bringing genomic analysis to the patient bedside to improve basic preventive measures (hand hygiene, environmental cleaning, catheter care, patient decolonization) via a user-friendly reporting platform. In preferred embodiments, this information can be filtered by hyper-transmissible, antibiotic resistant pathogens and those that display enhanced biofilm formation and reduced chlorhexidine susceptibility. The same information can be graphically displayed, and action plans recommended to target the specific virulence factors. The hyper transmissible, resistant, and biofilm producing organisms with reduced chlorhexidine susceptibility are targets for rapid diagnostic production and can be entered into the system. They are also targets for vaccine production.

Peri operative antibiograms can be provided that guide antibiotic selection using patient-specific as well as population level susceptibility patterns at the phenotypic and genomic level. Mismatches between genomic and phenotypic susceptibility identify emerging resistance. These isolates can be flagged and stratified by antibiotic class. Recommended antibiotics are those that the organism is susceptible to, the population is susceptible to at genomic and phenotypic levels, and there is the least amount of emerging resistance. This can improve antibiotic stewardship.

Preferably, the reports can include an interactive search option where a causative organism for an infection can be tracked, mapped, and targeted by these mechanisms. Empiric and prophylactic antibiotic choices are guided by the systematic biograms. Institutions can have a unique fingerprint such that infections that are reportable can be compared to the perioperative biogram to determine if representative of the institution, or likely derived from outside of the institution.

### EXAMPLES

### Example 1

Previous work by the Inventors isolated over 6,000 major bacterial pathogens from bacterial reservoirs. The isolates were obtained from serial surveillance of 2,170 environmental sites, 2,640 health care provider hands, and 1,087 patient samples during the process of patient care for 274 case-pairs, or 548 patients. From these reservoirs, more 6,000 potential and 2,184 true bacterial pathogens were isolated, including over 150 S. aureus isolates. Each of these isolates was archived, linked to a specific surgical case, to a specific day, to a specific operating room site, to a specific patient, and to a number of patient, provider and environmental demographic factors. Based on this archival, it was possible to begin the process of characterizing the epidemiology of bacterial resistance.

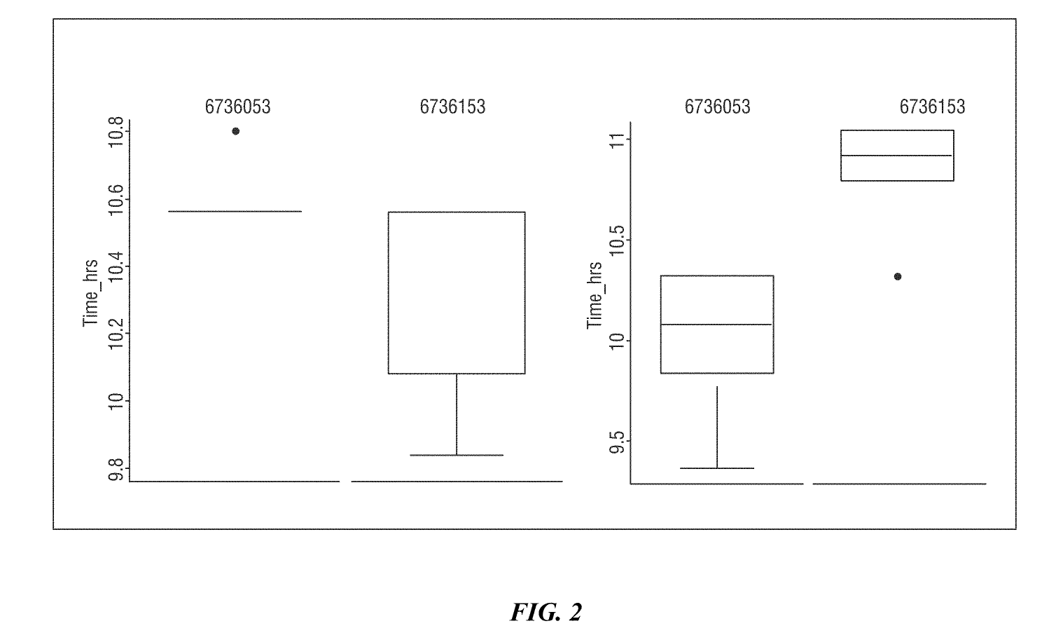

The first bacterial pathogen characterized was S. aureus, the leading cause of surgical site infections (SSIs). From the more than 150 S. aureus isolates acquired, clinical microbiology methods and laboratory approaches were used to classify these strains as having 9 different phenotypes among the MRSA strains and 18 different phenotypes among MSSA strains. Based on preliminary pulsed-field gel electrophoresis results, these strains represent approximately fifteen different S. aureus genotypes. Two of the phenotypes (U.S. Pat. Nos. 6,736,053 and 6,736,153) account for 59 and 70% of all MRSA and MSSA isolates, respectively. As compared to 6736153, phenotype 6736053 is more likely to be associated with resistance to methicillin [RR (risk ratio) 2.84, 1.40-5.73, p=0.008], resistance to ciprofloxacin (p=0.029), and infection (RR 1 1, 1.19-101, p=6.930). Furthermore, strains with this phenotype are more likely to be derived from patients than providers (RR 1 0.67, 1.3-2.15, p=0.014), and are more likely to be transmitted (operating room 2.02, 0.946-4.31, p=0.069). As compared to all other phenotypes, strains with the 6736053 phenotype are more likely to cause infection (RR 5.4, 1.15-2.07, v 0.049). Further, as shown in FIG. 2, 6736053-strains have an enhanced growth rate (a doubling time of approximately 45 minutes).

This is a significant finding, as evidence suggests that prophylactic antibiotics when administered as a bolus in the operating room, immediately prior to incision, take approximately 30 minutes to reach an effective minimal inhibitory concentration at the tissue level. Thus, a 30 minute growth advantage may be enough to allow sufficient growth for biofilm formation, quorum sensing, and enhanced bacterial defenses. This experiment was based on colorimetry where bacterial growth is detected by CO₂ production significant enough to cause a color change which is automatically detected and time-stamped. Finally, isolates with the 6736063 phenotype have significantly more beta hemolytic activity (p<0.001).

As such, sufficient infection risk information has been to obtained for clinically relevant S. aureus phenotypes with prognostic capacity and have further stratified isolates into virulent, likely to harbor or develop antibiotic resistance and/or to cause infection, and less virulent (less likely to have or to acquire antibiotic resistance and/or less likely to cause infection, groups. While the current standard is to classify patients S. aureus as being methicillin-resistant (MRSA) or sensitive (MSSA), our studies further refinement is now possible base on current patient screening strategies to identify patients with high-risk S. aureus phenotypes. These data also indicate that all MRSA and MSSA should not be considered equally but should be stratified according to virulence as described. This information can be used to develop and implement infection prevention measures targeting these high risk groups.

### Example 2

The entire process can provide detailed feedback to a quality assurance team on the performance of their preventive measures focused on attenuation of bacterial transmission and subsequent healthcare-associated infection development. The detail includes the source of bacterial infections (reservoir of origin), modes of transmission, key portals of entry to the patient, key portals of exit related to the patient, and key pathogen strain characteristics. This information is generated from a subset of patients identified as high risk by proprietary predictive modeling and applied to the population to effect global improvements. Outputs require integration of several data streams, including patient performance results and bacterial success, defined as clinically relevant pathogens.

The process includes pre-procedure patient data analysis, post-procedure patient data, reservoir collection and systematic-phenotypic processing of bacteria to identify epidemiologically-related bacterial transmission events and clinically relevant bacterial pathogens (transmitted, linked to infection, and/or a member of ESKAPE pathogens), information used to guide improvement at the group level, and whole genome analysis of clinically relevant bacteria to generate focused improvement strategies at the individual level, to identify structural variants/consequences, and to examine the impact of those variants on transmission, infection, and/or ESKAPE pathogen inheritance to develop novel diagnostics, disinfection agents, antibiotic therapy, and/or to generate improvements in existing agents.

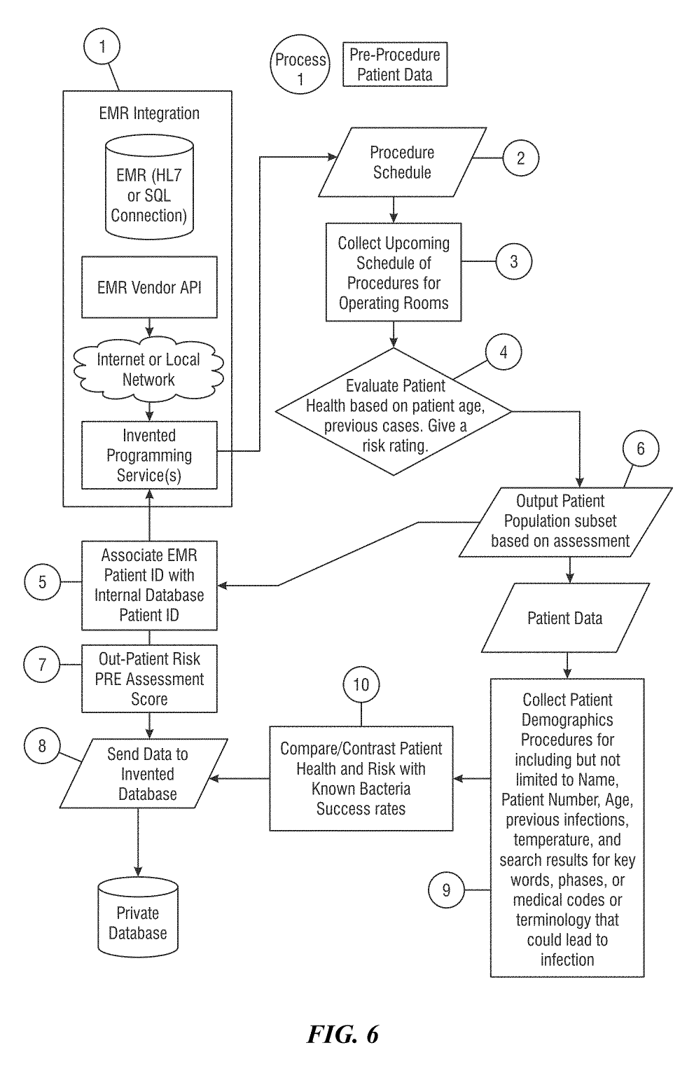

Process 1 is shown in FIG. 6. The system starts with Electronic Medical Records (EMR) integrated to the client system pulling patient data (1). This system connects either through SQL directly within a connected network, or VPN, SFTP, or through a vendor API. This connection changes depending on the EMR system it is connecting to at the time. Others may even require manual processes. (1). The system pulls the procedure schedule (2) and collects the upcoming operating room (OR) schedule (3). Calculating the risk of each patient (4) based on predefined and expanding criteria; it will determine a high risk patient population (6). This subset is the patient population that will be monitored to inform the entire perioperative patient population. The system will assign an internal patient ID to the patient, linked to the demographic number, case-log-ID, medical record number, and all associated barcodes generated from bacterial surveillance and store that in the Private Database. This associated patient ID is the key that will connect the patient within the two systems.

The system processes the subset population of patient data, automatically generates refined predictive modeling of factors defining group membership for those patients likely to become infected, and ultimately determines the patients (preoperative same day, preoperative emergency room, preoperative intensive care unit, and preoperative hospital ward) that need to be monitored by process 2. Identified patients trigger systematic surveillance by hospital employees that are trained by our group. This process requires that hospital information systems in each of the key preoperative patient arenas process patients as early as possible (7-10 days out for elective patients, immediately on hospital admission for other patients) using predictive scores. This data includes predefined criteria that will expand with the system, and it will be empowered to include search results for key words, phrases, or medical codes or terminology that could lead to infection (9), also see attachment A for additional key questions that will be explored as predictors with system implementation. It compares/contrasts this patient health data and risk to clarify high-risk group membership (10) and sends it to the invented data (8). The process assigns a Patient Risk Pre-Assessment Score (7) and stores that in the private invented database. A predetermined risk score is required for entry to process 2.

Process 2 is shown in FIG. 7A. The process continues to systematic preoperative, intraoperative, and postoperative bacterial reservoir collection. This part of the process includes general swab kits for each reservoir. The reservoirs collect from at least a set number of predefined points in a room, environment, on a person, object, or a mouth swab, hand, nose, pan, air temperature, body temperature, armpit, or rectal (11). By default, the reservoir collection is given standard priority and processed over a 48-72-hour period. If the operating room is in an alarm state, the collection media is marked as alarm-state and given a rapid response (11A). The rapid response process is handled by rapid, point-of-care diagnostics generated from genomic analysis for clinically relevant pathogens identified at each site. The results from rapid response require approximately 1 hour from swab to result and are entered into the system for automated, standardized outputs. In all other cases outside, the operating room employees test using the conventional swab kit(s) (12) and send the collection media to private lab for processing (13) which requires 48-72 hours for systematic-phenotypic results, and 7-14 days for genomic analysis results. For each sample, rapid or standard processing, random bar codes are generated that are linked to the manual input of demographic number, procedure date, and case-procedure-log-identification number (14, 15). The logged, stored bacteria are processed in the lab environment and location data is stored in the private invented database.

FIGS. 7B-F show reservoir collection software reporting, cataloging of hyper transmissible and resistant pathogens, cataloging of organisms with enhanced ability to form biofilm and reduced susceptibility to chlorhexidine, red arrow diagrams depicting the epidemiology of transmission of these factors. In all charts the operating rooms with higher likelihood of involvement are highlight, and then reports on patients involved in those transmissions with data from the model.

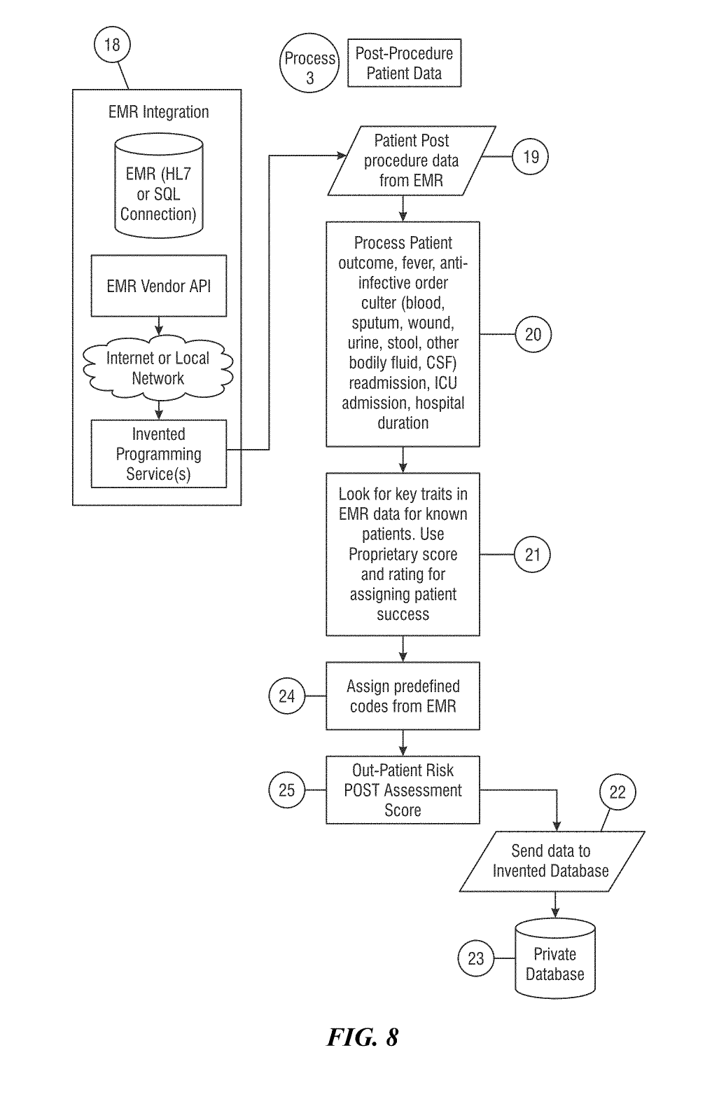

Process 3 is shown if FIG. 8. After the operating procedure, the system tracks patient performance by connecting to the EMR system (18), (19), similar to (1) or manual entry. The system analyzes patient outcome data including fever, anti-infective order(s), culture(s) (including blood, sputum, wound, urine, stool, or other bodily fluid) and postoperative visit note lacking the words “there is no sign of infection.” This process also evaluates for hospital 30-day readmission and hospital duration (20). Patients that are positive for 1 or more of 5 criteria above are identified as having a possible infection, and in any case of culture acquisition, the system generates an automated report for the clinical microbiological laboratory to save the cultures and send to the lab for further processing (21). In addition, the system assigns predefined codes that define the type of infection (blood, respiratory, wound, urinary, other) (24). The system assigns an overall post-assessment patient success score [infection is 1; no infection is 0, plus the code for the identified infection subset (25)]. This is similar to (7) and stores 19,20,21,24, and 25 data to the private invented database (22, 23). The information generated is linked to the manual input of demographic number, procedure date, and case-procedure-log-identification number (14, 15).

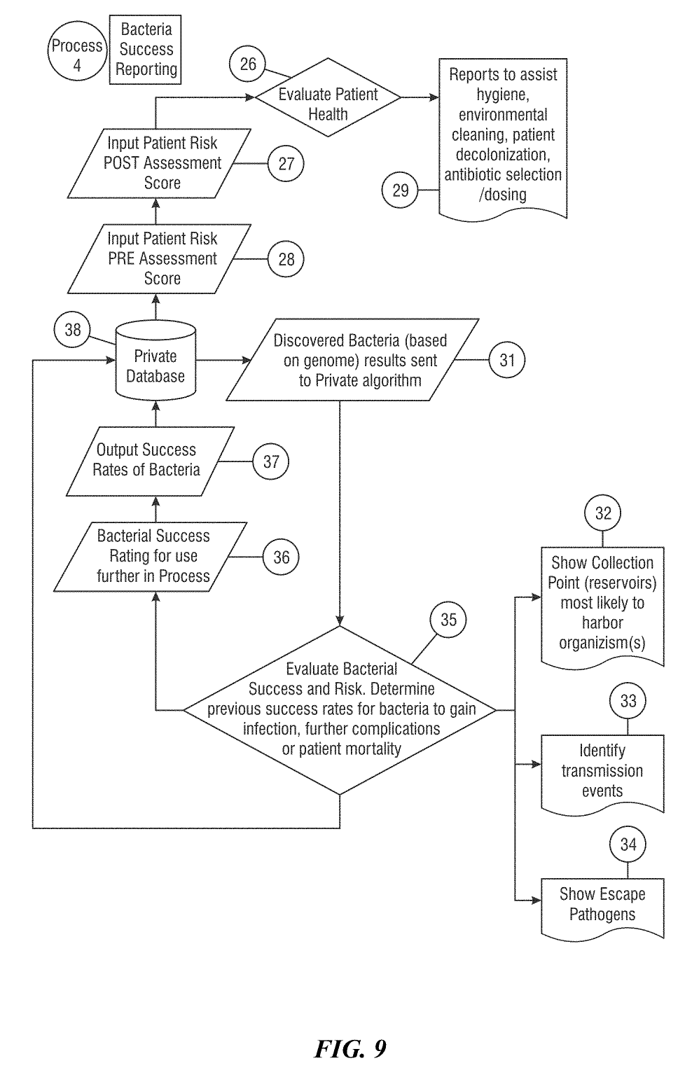

Process 4—Epidemiologically-related outputs—is shown in FIG. 9. This bacteria success reporting process(es) includes combining pre-(28) and post-(27) assessment success scores that evaluate patient risk of infection and development of infection (26) with data from process 2 that is analyzed in process 4 through a systematic-phenotypic analytical process to identify epidemiologically-related bacterial transmission events and pathogen strain characteristics that are associated with hyper transmissible, hyper virulent (more likely to infect), and hyper resistant (more resistant to antibiotics) organisms. Bacterial characteristics associated with overall poor patient health, infection development, hospital readmission, and/or increased hospital duration are summarized and used to assist in hygiene environmental, cleaning, patient decolonization, antibiotic selection and dosing (29) at group (process 4) and individual (process 5 levels). Any pathogen identified in this process as involved in an epidemiologically-related transmission event (ERTE), as an ESKAPE pathogen, and/or is linked to infection, readmission, or increased hospital duration is considered clinically relevant and moved into process 5. For all isolates processed, reservoir of origin, mode(s) of transmission, portal of entry/exit, and pathogen strain characteristics are summarized and reported every 48 to 72 hours.

Reservoirs shown most likely to harbor organisms indicate where the quality assurance team should give attention and sterilize more deeply. The reports showing transmission events indicate intersections between a high-risk patient and successful bacterial infection(s), as do modes of transmission and portals of entry. Strain characteristics provide insight into the traits that convey phenotypic success in a given environment, information that can guide improvement strategies and even basic science approaches involving complementation analysis. Reports showing escape pathogens provide tracking of the most dangerous bacteria today and keep pace with the acquisition of strain characteristics (antibiotic resistance, modes of transmission, phenotypes, etc.) that drive the success of those pathogens in a given hospital unit, between hospital units, between hospitals, within a region, between regions, and within a country.

Systematic-phenotypic analysis of the data (39) uses data from the patient data (44, 43), discovered bacteria location (46, 45), and bacteria success ratings (i.e. clinically relevant is 1, not relevant is 0) (49, 48). It updates the internal private invented database with the entry points, collection points and occurrences or intersections (47) for these bacterial isolates The system uses lab-collected data (50), and data based on previously known discovered bacteria characteristic(s) including genome and biological classification(s), to determine the following: 1) bacteria collection point(s); 2) bacteria transmission destination(s); 3) mode of transmission: within and/or between cases, specific procedures, days between; 4) route of patient entry (stopcock IV, skin, wound; 5) portals of exit (Foley); 6) pathogen strain characteristics; 7) outcomes of transmission (hospital, readmission, ICU); 8) predictors for recurrent transmission and identification of institutional reservoirs; and 9) predictors for measured outcomes (51). This data is summarized according to these automated outputs to be shared with infection control teams for focused improvement.

This information is then processed via proprietary analytics to identify and to characterize transmission events. Transmission reporting classification includes epidemiologically-related transmission events: the same class of pathogen present in more than one site within or between study units that has the same response to 7 biochemical reactions and the same response to 15 tested antibiotics (the same BugID). Reports are generated every 48 to 72 hours, but because reports are continually being generated, the end user has real-time access at any time to what any major pathogen is doing

Process 5-Clonal outputs—is shown in FIG. 10. The process inputs data from the reservoir collection (30) and the internal private invented database and sends the discovered successful (clinically relevant) bacteria to a private algorithm that identifies insertions, deletions, and single nucleotide variants that associate with bacterial success (31). It also compares sequences of isolates involved in epidemiologically-related transmission events to identify clonal transmission events, which is then analyzed to characterize the epidemiology defining those events, and to identify clinically relevant (refined) structural variants and consequences at the nucleotide level that identify new, optimal targets for diagnostics and therapeutics to inhibit bacterial transmission, virulence, and resistance (35). This data is then assigned a success rating and for use further in the process (36). The bacteria success rating is stored in the private internal invented database (37) that is searchable by bacterial class, phenotype, genotype, single nucleotide variant and functional consequences.

Reports are available to show the collection point (reservoirs) most likely to harbor clinically relevant or other organism(s) (32), to identify transmission events and locations of those events, modes of transmission, portals of entry/exit, strain characteristics (33), and to show escape pathogen involvement/presence (34).

As above, it remains true that reservoirs shown most likely to harbor organisms indicate where the quality assurance team should give attention and sterilize more deeply. In this case, however, the reports are much more definitive and can, for example, identify specific provider involvement, whereas epidemiologically-related outputs would identify provider group level involvement. It is also true that the reports showing transmission events indicate intersections between a high-risk patient and successful bacterial infection(s), as do modes of transmission and portals of entry. In this case, however, these are much tighter associations; they are nearly irrefutable intersections that can be addressed with confidence. It is also true that strain characteristics continue to provide insight into the traits that convey phenotypic success in a given environment, information that can guide improvement strategies and even basic science approaches involving complementation analysis. However, in this case we generate associated information regarding structural variants and consequences, leveraging next generation sequencing, yielding the potential for construction of rapid, point-of-care diagnostics that can maximally attenuate their spread, in alarm states, or even more globally. Reports showing escape pathogens continue to provide tracking of the most dangerous bacteria today, but in this case, they keep pace with the acquisition of genetic traits that drive the success of those pathogens in a given hospital unit, between hospital units, between hospitals, within a region, between regions, and within a country.

Genomics analysis of the data (39) uses data from the patient data (44, 43), discovered bacteria location (46, 45), and bacteria success ratings (i.e. clinically relevant is 1, not relevant is 0) (49, 48). It updates the internal private invented database with the entry points, collection points and occurrences or intersections (47) for these bacterial isolates The system uses lab-collected data (50), and data based on previously known discovered bacteria characteristic(s) including genome and biological classification(s), to determine the following: 1) bacteria collection point(s); 2) bacteria transmission destination(s); 3) mode of transmission: within and/or between cases, specific procedures, days between; 4) route of patient entry (stopcock IV, skin, wound; 5) portals of exit (Foley); 6) pathogen strain characteristics; 7) outcomes of transmission (hospital, readmission, ICU); 8) predictors for recurrent transmission and identification of institutional reservoirs; and 9) predictors for measured outcomes (51). This data is summarized according to these automated outputs to be shared with infection control teams for focused improvement.

This information is then processed via proprietary analytics to identify and to characterize transmission events. Transmission reporting classification includes: 1) Clonal transmission events: epidemiologically-related events that have sequences with >95% identity, the same response to multi-loci sequence testing, and/or are identified with rapid, point-of-care diagnostics that are based on single nucleotide variant analysis using real-time polymerase chain reaction technology, and 2) Institutional reservoirs: isolates involved in clonal transmission events are found across days, weeks, months, and/or years between study units (52). Reports are generated every 7-14 days for sequencing analysis, or within one hour where possible or in an alarm state via use of rapid diagnostics.

### General Processing

In all cases of transmission events, epidemiologically-related or clonal, the isolates are considered clinically relevant and are characterized individually and at the group level in terms of pathogen strain characteristics, including structural variants at the genomic level, and according to the epidemiology of the transmission events (mode, reservoirs or origin, portals of entry/exit, transmission locations) (54). In all cases of ESKAPE pathogens, this same processing is conducted and reported. Additional reporting will be created as needed assisting with causation and correlation (53).

The lab processing for any culture swab starts with a physical specimen that is cataloged (56). The user checks for alarm state to assign priority (57). The system refines epidemiologically-related transmission events (ERTEs) and identifies ESKAPE pathogens (58). This data is sent to the lab workflow (59). The lab workflow (59) first creates a subculture over 24 hours (60). It makes a glycerol stock to store relevant pathogens in the archive and assigns a freezer location (61). It uses 0.5 McFarland standards to generate DNA Stock Solution (62). It extracts the DNA (63). It Quantifies and Qualifies the DNA (64). It uses MiSeq sequencing generating sequence results (65). If there is an alarm state it includes rapid, in vitro, point-of-care diagnostic tests that are available (66). FASTO files are uploaded to CLC Genomics Workbench (70). Isolates are then compared via whole genome analysis to the nucleotide level. If they are greater than 95% identical they are considered clonal and are simply identified as such. The system runs analytics on the outputs, but with enhanced strength to pinpoint the source (71) and assigns an identity to the bacteria (73).

Even if they are not clonal, they remain clinically relevant, and they move on for further analysis (75). This analysis (shown in FIG. 13) identifies a best reference by K-mer analysis, aligns sequences to the best reference, realigns based on structural variants, insertions, and deletions, identifies single nucleotide variants via fixed ploidy analysis, assesses functional consequences of variants including amino acid changes, assesses impact on 3D protein conformational change, annotates variants with flanking sequences, and generates outputs to meta tables, with all variants and associated information linked to unique IDs. The system updates the internal private invented database with this DNA data or identity and is reanalyzed to identify successful groups (phenotypes based on biotype or antibiotic resistance profiles, genetic traits common within or between pathogen groups) or specific isolates or strain characteristics/traits, with success defined by links to infection, hospital duration, readmission, or even death (68).

The system takes the input of the data, bacteria characteristic(s), classification(s), systematic phenotypic processing (78), and loops through analyzing at the nucleotide level for insertions, deletions, at the chromosomal level, plasmids, epigenomic information (82). The system compares and contrasts known bacterial classes and characteristics with discovered bacteria to determine if it is previously known or a new mutation (83). All mutations are categorized according to pathogen, position, and functional consequences and are linked to all clinical data for relevance, searchable by unique ID. This is the searchable interface for targets for development of novel diagnostics/therapeutics. Possible user input of genomics data and transmission events are required (87). The identity is stored in the private internal invented database (88). If required, the system assigns an identity to the new bacteria (89) and updates the database with the genetic data (81).

Reporting generated on request will include targets of relevant genetic variants, transmissibility, virulence, resistance, and other traits (86). With user input, we will generate these outputs in order to build diagnostics or to compare variants within groups (74).

Process 6

All structural variants of clinically relevant pathogens undergo extensive processing to determine the clinical relevance (infection, readmission, hospital duration, and death) in order that the most relevant targets can be identified, analyzed, and utilized to advance patient safety.

What is claimed is:

1. A method of preventing bacteria transmission comprising:
identifying patients at risk of developing postoperative infections, wherein said identifying comprises:
obtaining infection risk information from one or more pre-operative, intra-operative, or post-operative arenas;
screening patients for development of or as having a high risk for development of infection, wherein said screening comprises processing the infection risk information; and
identifying one or more patients as being at risk for a particular post-operative infection based on said screening; and
providing one or more patients identified as being at risk for a particular post-operative infection with one or more treatments capable of treating or preventing said infection;
obtaining physical samples from environment, patient, hand and other samples from the identified high risk patient, operating room, and hospital ward;
wherein the method results in infection reduction.

2. The method of claim 1, wherein said identifying one or more patients further comprises generating an alert, and delivering said alert to a healthcare provider.

3. The method of claim 1, wherein said processing comprises predictive modeling; and wherein said screening comprises an automatic process of detecting patients who develop infections and/or require the acquisition of patient cultures for assessment of infection.

4. The method of claim 1, further comprising: (a) using a collection kit for ordered reservoir surveillance and (b) reporting on the reservoirs.

5. The method of claim 1, wherein said assessment and identification is performed by software that identifies one or more of the following:
patients that become infected;
ESKAPE and other bacterial transmission events;
epidemiology of bacterial transmission events;
genetic clonal transmission events;
whether bacterial transmission events are linked to infection development; and
clinically relevant bacterial pathogens.

6. The method of claim 5, wherein said clinically relevant bacterial pathogens are bacterial isolates that are hyper transmissible, hyper virulent, and/or hyper resistant to antibiotic therapy.

7. The method of claim 5 further comprising identifying a group of bacterial isolates and cross-referencing the bacterial isolates with test results to determine transmitted phenotypic bacteria.

8. The method of claim 7 further comprising performing genomic tests on the bacteria isolates and identifying a subset of bacteria by genomic properties that were originally identified.

9. The method of claim 1 further comprising evaluating clinically relevant bacterial isolates to identify structural variations in said bacterial isolates, wherein said evaluation identifies variants that are unique to clinically relevant pathogens.

10. The method of claim 9, wherein said evaluating comprises the steps of:
collecting environmental or patient samples;
producing bacterial isolates from said samples;
determining the bacterial class of one or more bacteria derived from said bacterial isolate;
determining the biotype of bacteria derived from said bacterial isolate;
determining a specific sequence of antibiotic susceptibility, and
comparing two or more bacteria derived from different bacterial isolates to assess epidemiological relation defined by the same specific sequence of antibiotic susceptibility.

11. The method of claim 10, further comprising producing an archive sample by:
culturing bacteria derived from a bacterial isolate;
extracting DNA from the bacterial culture;
sequencing the extracted DNA;
analyzing the extracted DNA, comprising
identifying single nucleotide variants, deletions, and insertions in the extracted DNA;
evaluating protein conformational change produced by identified nucleotide variants, deletions, and insertions;
evaluating the impact of identified nucleotide variants, deletions, and insertions impact on drug binding sites.

12. The method of claim 11, further comprising comparing sequence identity to determine if a clonal transmission event has occurred, wherein clonal transmission >95% similarity of sequences and the same output from multi-loci sequence testing indicates a clonal transmission event.

13. The method of claim 12, further comprising producing a bacterial archive comprising two or more archive samples.

14. A system for screening patients for development or having a high risk of development of infection, comprising:
an information system in one or more of a pre-operative arena, an intra-operative arena, and/or a post-operative arena, wherein the information system automatically processes patient demographic information;
a laboratory-based surveillance system;
a computational system for identifying bacterial transmission events, the epidemiology of bacterial transmission events, patients that become infected, whether bacterial transmission events are linked to infection development, and bacterial isolates that are hyper transmissible, hyper virulent, and/or hyper resistant to antibiotic therapy (clinically relevant bacterial pathogens); and
a database comprising bacterial isolate identities and bacterial isolate traits, wherein database is linked by an interface for communicating with a computing device to provide information relating to one or more of the other systems.

15. The system of claim 14, wherein the database comprises a multiplicity of sample storage devices, wherein said sample storage devices are configured to store bacterial isolates, and wherein said sample storage devices are operatively connected to an interface for communicating with a computing device to provide information relating to a bacterial isolate contained in the sample storage device.

16. The system of claim 14, wherein the system comprises information about patients, a pre-operative arena, an intra-operative arena, a post-operative arena, a healthcare provider, or a combination thereof.

17. The system of claim 16, wherein the surveillance system provides real-time, continual surveillance of ESKAPE bacterial transmission events.

18. The system of claim 17, wherein the surveillance system provides reports to a healthcare provider.

19. The system of claim 16, wherein said system identifies patients who develop infections and/or require the acquisition of patient cultures for assessment of infection.

20. A method of making an archive of bacterial isolates, comprising:
identifying patients at risk of developing postoperative infections, wherein said identifying comprises:
obtaining infection risk information from one or more pre-operative, intra-operative, or post-operative arenas;
screening patients for development of or as having a high risk for development of infection, wherein said screening comprises processing the infection risk information; and
identifying one or more patients as being at risk for a particular post-operative infection based on said screening; and
producing archive samples, wherein said producing comprises:
collecting environmental and/or patient samples;
producing bacterial isolates from said samples;
determining the bacterial class of one or more bacteria derived from said bacterial isolate;
determining the biotype of bacteria derived from said bacterial isolate; and
comparing two or more bacteria derived from different bacterial isolates to assess epidemiological relation;
culturing bacteria derived from the bacterial isolates;
extracting DNA from the bacterial cultures;
sequencing the extracted DNA;
analyzing the extracted DNA, comprising
identifying single nucleotide variants, deletions, and insertions in the extracted DNA;
evaluating protein conformational change produced by identified nucleotide variants, deletions, and insertions;
evaluating the impact of identified nucleotide variants, deletions, and insertions impact on drug binding sites;
compiling said archive samples to produce a bacterial archive, wherein said archive samples are linked to data acquired in any of the preceding steps.
